SEQUENCE LISTING (23 sequences)

SEQ ID NO: 1 - DNA, 2487 bp, Sarocladium sp.
atgaagtctcccagtctggccgtggccggccttcttcttggctcgagttccttaagccatgctacccagcttcgtattgagacgaggaaatcgctcaactctcgtatcgccaacgtccacattgatgttgacgctccagtcgctcaccaagttgtcttcacatatggcccttgtgattcggagagccaagagaacgctcaccatgtcattgctcaaagccaaaagctagagggcaggaaaccccatcgattgatctggactatgcccaaggatcttcacccggatgactgcatctctgcgtggggtgaatcaggagacctcctcggtcgcagtgtccctcaaaaggtcgcgcacaaggagatgaggagacgcaagagagatgatagcgactactccatccctatgaacagctcgagtggcatcgacgtttatggcccttggttcgatggtgtagctctcctcgagaagagcgacaatcacaatgtggacgtcgaagccgccaaggccaaggagattgctatcgtcggggctgggatggctggtctgaccacgtacttcatcctcagtgaagctggactcagcaacctgacgatcctggaggcaagtggtcgtcttggaggccgcgtacgcaccgaatatctctccggaggacccagggaccattcctatgccgagatgggccctatgcggatcccataccaggctcgttttggagacaaggcatacaacatctcggatcaagcaatattcttccgacttgtggagaaggtgaacgagcggaacaagaagcttggaaacaccaaggacctcatcaacctgatcccgttcatccagtccagtccgaatgggctcgcttattatcaaggtaacaagctggagaatgggctgccgccaacgcaggcagatgttgctgcagatccagcactggggaatgaatcccctgagatccctgaatctgcacaagaattggctgctcagcttcagagagcattgcctaacgctgaattccttgagctaatggccacaaacttctggcaagcccatgccgaattcctcggtatgttgcttcagctcaccaccccgtatagccagggctctcaaaccctcatagaggatgccactaaaatgaaatgcgaatgtagagaatcagggtccagctggcctgcctggcgatcagtggtctgagtttgctttcctggtgaactacctcaacgcgaccgtcttcgatgccaacgcagtcactggtggttatgactggcacagcagccttgacagggtacgtgacgctgaagaagatctcgtggttgcatcattaatcccacggtctccctagttgtactacactatgctcttcggaggcgggggcagtttcaagacgattgatggaggcaagtcaaaatgagcgttgcgggcacctggacgagaactgacagagctcgctcgtaggcatgaacttgctccccaacgctttccatcccctagttgacgatatcacgaagttcaatgcaaaagtagagaaggttcagctcgatgagaagacttcccgcctgaagctgcattggcgcgcgaactacactgatccggagctcgagtcgcagtcatttgactacgccattctgtcacctaccatgccagcagtacagaagttgcgccttccaggtaggtcctgcgcaatctctcgttgctttgcggaccatagctttcaatgtcacaggtctacccttcgctatgcgcaacgccgtcgactccatgccttacgcctctgcgtgcaaagtcgccctcgaataccgcactcggttctgggaaaagttcgacaaccccatctacggctcctgttccaccagcaccgacattcccggtattggatccgtgtgctacccatcttccaacatcaacggcagcggcccagcttccctccttgcgagctacgagattggcaggccttacggcgcagagtgggctggcattcctgaggagcagcatgtgcagtatgtgattgacgcaatgattgatatccacggcgaggttgcacgacgagagtttaccgggaaatgcaagagaaagtgttgggctctggacgagttctcaaacgggggttgggcttcaccgaccgtgggtaatcatgagacgtatctgccatcgtttttcgagacccacagccatgtgagtgctccgacatttgacctcttgtgcgaggaatgagggacttgttgaccttgttccagatgatattcgtgggtgagcatacctcctatacacacgcatggatcgcctctgcgatcgaatctgcagtccgaggcagcgtgcagctgctcttgggtatgtttcgcgttcttgcaaatcgtgcagatgtgcttctgacattactttgccacagagcttggccttatagacgaggcgaaggatgtcgtaaacacgtggatggcacgatggatcagtgttgtaagtgacctaagcaaagccgtggtgtctggagcgtcagcgtag

SEQ ID NO: 2 - protein, 689 aa, Sarocladium sp.
MKSPSLAVAGLLLGSSSLSHATQLRIETRKSLNSRIANVHIDVDAPVAHQVVFTYGPCDSESQENAHHVIAQSQKLEGRKPHRLIWTMPKDLHPDDCISAWGESGDLLGRSVPQKVAHKEMRRRKRDDSDYSIPMNSSSGIDVYGPWFDGVALLEKSDNHNVDVEAAKAKEIAIVGAGMAGLTTYFILSEAGLSNLTILEASGRLGGRVRTEYLSGGPRDHSYAEMGPMRIPYQARFGDKAYNISDQAIFFRLVEKVNERNKKLGNTKDLINLIPFIQSSPNGLAYYQGNKLENGLPPTQADVAADPALGNESPEIPESAQELAAQLQRALPNAEFLELMATNFWQAHAEFLENQGPAGLPGDQWSEFAFLVNYLNATVFDANAVTGGYDWHSSLDRSSLVGMNLLPNAFHPLVDDITKFNAKVEKVQLDEKTSRLKLHWRANYTDPELESQSFDYAILSPTMPAVQKLRLPGLPFAMRNAVDSMPYASACKVALEYRTRFWEKFDNPIYGSCSTSTDIPGIGSVCYPSSNINGSGPASLLASYEIGRPYGAEWAGIPEEQHVQYVIDAMIDIHGEVARREFTGKCKRKCWALDEFSNGGWASPTVGNHETYLPSFFETHSHMIFVGEHTSYTHAWIASAIESAVRGSVQLLLELGLIDEAKDVVNTWMARWISVVSDLSKAVVSGASA

SEQ ID NO: 3 - DNA, 2086 bp, Sarocladium sp.
atgttcactcccaaagcttggatccctctgttggccttgagccgcgaggtcttctccaatcctacttctgcatcccattccatctctttccacggtctccttggcgtctcttcggagtcagtccacaacatccacctcacttatggcgatgccttcccacatggagacttccgtgttgtctttggagactgcggtatgaccagtgaggatgaacttcatcacgaggttgcatctctctccacgaagatggagtctgctcctgatcgtttggtctggcttgtgccaaaggatgtccgcgagaatggttgtcttcacgctttctcggagggcgtcctcctcggtcggtctgagcctgtcgctttcacagagccactccggaagcgcgagtctctctctgagattggtgactccgatggcctctggttcagcggaatccgctatctgcagtcaagcaacctgacatccgtcaaggccgccgaagctaaggaaaagaagattggcatcgttggcggtggcatttccggtctgatgacgagtctgctcctgacttccgtcggcatgaccaactggcacatcattgaggcgactgagcgtgtcggaggccgcatccgcaccaagtatatgaatggaactagtcccgatgactaccagtaccaggagatggggcccatgaggttccccgttgaggtcaaatacaacgacaccaacgagacgctgcccattcaggatcacaagatggtcttccagctggctgaagtcttgaacgagatgaacggcaacgacacagatctcgctgtcaagttcattccctggacccagaacaatccgaacactcccgccaactctcgaggataccgcctccctgatggccgcatccctaccgcggcccaaatcgcccagaaccccagcatcttgccggcagctgccaacgcgtcagacccccaagatgcggaacttggcaaggagtccttggagcgtctcagtgaccttacccccgagcggatgcgcaacatctccgccaacatcttcaaggcgcaccgagatgccattgaccgcggcttgtttcactggtctgaggcagcctatctccggtaccagctgggccttgacgacgatactgtggacttcgtggctgcctccgacaactatcccatgttccctgactggtggcacgcggtctacttcgcggccacgaagtggctcacgatcgacaagggcctcgattcgctgtccagggcttttgtaccccacgtcaaggacaagatcacgtacggtcgcaagattgaagccatgcaatggaatgagtcaacatccaagatctcactgtcatggagggagagccctcttgcggcggcgaagtccgatgagtacgactatgctgtcgtcgcggtgccgttctccaaggttcgtctgtggaagcggccggcttactccaacctcttgacgagggctattggcaagctgaactacgagcaggcctgcaaagtgagcaccccctcatccgcccgaagagtgactagaatactaatccctccaaaaggttgctctgctctacgagacacgcttctgggagcaccaggagatccccatctttggaggctgtggctcggttgatatcccgggtattggtggcgtgtgctacccatcatacgagatcaactccacgaggcctggcgtgattctctcttcctacattaccggcacagaagccagatccgtggtagccctgagcgaggaagatcacgtcgccatggtgcagcgtgccatggtcgaagtccacggccctatggcggatgagcagtggaccgggatttacgaccgtctgtgctgggaggtggatgagaacgcggccggaggctgggcttcgccgacagttggccagcaggagctgttcatcccggcgtaccacaagacggagctcaacaccatcttcatcggagagcacacgagcatcacgcacgggtggatcttctcggccctggaatcgtcggtcaggggcacgacgcagttgctgctcgatcttggtctagtggatgaggcgaagcagattgttgagacttggatggcgaggtggatcaccgtttga

SEQ ID NO: 4 - protein, 676 aa, Sarocladium sp.
MFTPKAWIPLLALSREVFSNPTSASHSISFHGLLGVSSESVHNIHLTYGDAFPHGDFRVVFGDCGMTSEDELHHEVASLSTKMESAPDRLVWLVPKDVRENGCLHAFSEGVLLGRSEPVAFTEPLRKRESLSEIGDSDGLWFSGIRYLQSSNLTSVKAAEAKEKKIGIVGGGISGLMTSLLLTSVGMTNWHIIEATERVGGRIRTKYMNGTSPDDYQYQEMGPMRFPVEVKYNDTNETLPIQDHKMVFQLAEVLNEMNGNDTDLAVKFIPWTQNNPNTPANSRGYRLPDGRIPTAAQIAQNPSILPAAANASDPQDAELGKESLERLSDLTPERMRNISANIFKAHRDAIDRGLFHWSEAAYLRYQLGLDDDTVDFVAASDNYPMFPDWWHAVYFAATKWLTIDKGLDSLSRAFVPHVKDKITYGRKIEAMQWNESTSKISLSWRESPLAAAKSDEYDYAVVAVPFSKVRLWKRPAYSNLLTRAIGKLNYEQACKVALLYETRFWEHQEIPIFGGCGSVDIPGIGGVCYPSYEINSTRPGVILSSYITGTEARSVVALSEEDHVAMVQRAMVEVHGPMADEQWTGIYDRLCWEVDENAAGGWASPTVGQQELFIPAYHKTELNTIFIGEHTSITHGWIFSALESSVRGTTQLLLDLGLVDEAKQIVETWMARWITV

SEQ ID NO: 5 - DNA, 2070 bp, Sarocladium sp.
atgaagtctccatctctggctgtggctggtctcctgcttggatcctctagcctctcgcatgccacccagctgcgcatcgaaactcgtaagtcgctcaactcccgcatcgctaatgtgcatatcgatgttgacgcccctgtcgctcaccaggtcgtgttcacctacggtccctgcgattccgagtctcaggaaaacgcccatcacgttatcgctcagtcgcagaagctcgagggacggaagcctcatcgcctgatctggactatgcctaaggatcttcaccccgatgactgtatctctgcctggggagaatctggtgacttgctcggccggtccgtgccacagaaggttgctcataaggagatgcgccgtcggaagcgcgatgactctgactacagcatcccaatgaactcgtcctctggaatcgatgtgtatggcccgtggttcgacggtgttgccctgcttgagaagtccgataaccacaatgttgacgtcgaagccgctaaggccaaggagatcgctatcgtcggagccggcatggctggccttaccacttatttcatcttgtcggaagccggtctttccaacttgaccatcctcgaagcttctggacgcctgggcggtcgtgtgcgcactgaatacctttctggaggcccccgtgatcacagctatgccgagatgggccctatgcgtatcccctaccaggcccggttcggtgataaggcttataatatcagcgaccaggctatcttcttccgcctcgtcgagaaggttaacgagcgcaataagaagcttggaaacaccaaggacctgatcaatcttatccctttcatccagagctcgcccaacggactggcctactatcagggcaacaagttggaaaatggtctccctcccactcaagctgatgtggctgctgacccagctctcggtaatgagtcgccagaaattcctgagtctgctcaagaattggctgctcagcttcagcgtgctttgccgaacgctgaattccttgagttgatggccaccaatttctggcaggcccatgctgaattcttggagaaccagggtccagctggactcccgggcgatcagtggtctgagttcgccttcctcgtcaactacctgaatgctaccgttttcgacgccaatgctgtcactggtggatatgattggcattcctctcttgaccgcagctcgttggttggcatgaacttgctcccaaatgccttccacccgttggtcgatgacatcaccaagttcaacgctaaggtcgaaaaggtgcagctcgatgagaagacctctcgcctcaagctgcactggcgtgccaattacactgatccagagttggaaagccagtcgttcgactatgccatcctcagccctaccatgcccgctgtccagaagcttcgtttgccaggcttgccgttcgccatgcggaacgctgtcgactctatgccttacgccagcgcttgcaaggtggctcttgaatatcgtactcggttctgggagaagttcgataatcccatctacggttcctgctctaccagcactgacatcccaggtatcggatccgtttgttatccgtcctctaacatcaatggctccggtccggcctctctgcttgctagctacgaaattggacgtccttatggtgctgagtgggctggcatccccgaggaacagcatgttcagtacgtcatcgatgccatgatcgacatccacggagaagtcgctcgccgtgagttcaccggcaagtgcaagcgtaagtgttgggccctggatgagttctctaacggcggttgggcctctccaaccgtgggaaatcatgaaacttaccttccgtcgttcttcgagactcattcccacatgatcttcgttggcgaacatacctcgtatactcacgcctggatcgcctcggctatcgagtccgctgtccgtggttccgtgcagttgctcctggaactcggactgatcgatgaggccaaggacgttgtcaacacctggatggctcggtggatctcggtggtttccgatctgtctaaggccgtcgtgagcggtgcctcggcttga

SEQ ID NO: 6 - DNA, 2031 bp, Artificial Sequence
  codon-modified g4462 (penox1)
atgttcactcctaaggcttggattccactcctggctctgagccgcgaggtcttctcgaaccctacctctgcctctcatagcatctcgttccacggacttttgggcgtctcctctgaatctgtgcataatatccaccttacctacggtgacgctttcccccatggagatttccgtgtcgtgttcggtgactgcggaatgacttctgaggatgaactgcatcacgaggtcgcctccctttctaccaagatggaaagcgctccagaccgcctcgtgtggctggttccgaaggatgtgcgtgagaacggatgtttgcacgccttctctgaaggcgttctcctgggtcggagcgagcctgtcgctttcactgaacccttgcgcaagcgtgagagcctctcggaaatcggagactctgatggcctgtggttcagcggcatccgctatctgcagagctcgaatcttacctctgtcaaggccgctgaggccaaggaaaagaagatcggtatcgtgggcggtggaatctccggacttatgacctctcttttgctcactagcgtgggtatgaccaactggcatatcatcgaggctaccgaacgcgttggcggtcggatccgcactaagtacatgaatggaaccagccctgatgactaccagtatcaggagatgggccctatgcgtttccccgtcgaggtgaagtataacgacaccaatgaaactctccccatccaggatcacaagatggtgttccagttggccgaggttctcaacgaaatgaacggcaatgacactgatttggctgtcaagttcatcccatggactcagaacaatcctaacacccctgccaatagccgcggctaccgtctcccagacggtcgtatcccgaccgccgctcagatcgctcagaacccatctattttgcctgctgctgctaatgcttctgacccacaggatgctgagcttggcaaggagtcgcttgaacggttgtccgatctcactccggaacgtatgcggaacatctcggccaatatcttcaaggcccatcgtgacgctatcgatcggggtctgttccactggtccgaggctgcctaccttcgctatcagctcggactggatgacgataccgtcgacttcgtggctgcctccgataactacccaatgttcccggactggtggcatgctgtgtatttcgctgccaccaagtggctgactatcgacaagggcctggattccctttctcgggccttcgttccacacgtcaaggataagatcacttacggtcgcaagatcgaggctatgcagtggaatgaaagcacctcgaagatctccctttcctggcgtgagtcgccgctcgctgctgctaagtccgacgaatacgattatgccgttgtcgctgttccattctctaaggtccgtctgtggaagcgtcctgcctattccaacctgcttactcgcgctatcggcaagcttaattacgagcaggcctgcaaggtggctttgctctatgaaacccgtttctgggagcatcaggaaatcccaatcttcggaggctgcggatcggtggatatccctggcatcggtggagtttgttacccctcttatgagatcaacagcacccgtccgggagttatcttgtcctcttatatcaccggcactgaagcccggtcggtggttgctctctccgaggaagaccatgtggccatggttcagcgggctatggttgaggtccacggtcccatggccgatgaacagtggactggaatctacgaccgcctctgttgggaagtcgatgaaaatgctgctggcggttgggcttctcctactgttggacagcaggagttgttcatccccgcttatcacaagaccgagctcaatactatcttcatcggcgaacatacctcgatcactcacggttggatcttctccgccctggagagctcggttcgtggcaccactcagctgcttttggaccttggtttggtcgatgaggccaagcagatcgtggaaacttggatggctcggtggatcaccgtctga

SEQ ID NO: 7 - DNA, 748 bp, Artificial Sequence
  Ptef
tgtggaccagacaggcgccactcggccgggccacaactgcttgggttttgaccgggagcggaccaattaaggactcgaacgaccgcggggttcaaatgcaaacaagtacaacacgcagcaaacgaagcagcccaccactgcgttgatgcccagtttgtctgtccgaaatccaccggaaaggtggaaacatactatgtaacaatcagagggaagaaaaattttttatcgacgaggcaggatagtgactgatggtggggtcatggtcgggtctccgagcgaaagagaaccaaggaaacaagatcaacgaggttggtgtacccaaaaggccgcagcaacaagagtcatcgcccaaaagtcaacagtctggaagagactccgccgtgcagattctgcgtcggtcccgcacatgcgtggtgggggcattacccctccatgtccaatgataagggcggcggtcgagggcttaagcccgcccactaattcgccttctcgcttgcccctccatataaggattcccctccttcccctcccacaacttttttcctctttctctcttcgtccgcatcagtacgtatatctttcccccctacctctttctcactcttcctcgattcattccactcttctccttactgacatctgttttgctcagtacctctacgcgatcagccgtagtatctgagcaagcttttttacagaatctttctagtatcttacaaagaactacaaagttcgcaccaccttcaaa

SEQ ID NO: 8 - DNA, 800 bp, Artificial Sequence
  Talp
gtaccaggagtacattggagagttctaccattgttgctggaatacaatgatgattagaaaccgaagagtgttatgattcggacggatatacgcatggcacgcatacagcgtgatacataggctgtttgctcaagaattaggattttatctgaatccatgtacagagtttacttatgttagtagtcaatgaaatcttggctttctaattttgtccgatctacaaggggtagtcgatcacagaacgaactagatgtgcagggaacgatgatcacccgctcttagcaagacctctagtagttttcgaccatagctttaacgcgaatcatgaccctactattttctagattgcagaccaagtcacatgacaatgtcctctttgaagtaggatcagtagctgattagattccgggaaatgaattagggctggcgttccaactactggggagtgccgatgttgctgtatgaaagatagtaagattactagtgcacagctgtagtaattatttactctagattatatattccaaataataagtaatctaagatagtagacagtcctatgatatagctccgggttcgaagtcggcaaaagatatgcaatcacctgtcgggatgatatatgtatatctgaaataccgacatcaaccatccagtcggatcagctaaacgaagtatcacttctttcgccactgccaatcactacttctattaaagttcatgttacagtataagccacaagacttatctccagaactaacttgtgcataggagctctgccgatagccgggtggttggatcgg

SEQ ID NO: 9 - DNA, 1487 bp, Artificial Sequence
  pyrG3
taagtactcatttatacaatagttgcagaaccccgcgctacccctccattgccaacatgtcttccaagtcgcaattgacctacagcgcacgcgctagcaagcaccccaatgcgctcgtgaagaagctcttcgaggttgccgaggccaagaaaaccaatgtcaccgtttccgccgacgtgacaaccaccaaagagctgctggatttggctgaccgtatgcgcaccggggatgccacttacatatgatctagtaatggttaatggtggaatatataacaggactcggtccgtacattgccgtgatcaaaactcacatcgatatcctctccgatttcagcgaagagaccatcatcggtctgaaggcccttgcagagaagcacaatttcctcatcttcgaagatcgcaagttcatcgatatcggaaacacagtccaaaagcagtaccatggcggcactctgcgcatctctgagtgggcccacatcatcaactgcagtattctgcccggtgagggtatcgtcgaggctctggcccagactgcttcggccgaggacttcccctatggctctgagaggggccttttgatccttgcggagatgacatccaagggatctttggctaccggtcaatatactacttcttctgttgactatgcccggaagtataagaagtttgtgatgggattcgtctcgacgcgtcacctgggcgaggttcagtctgaagttagctcgccttcggaggaggaggatttcgtcgtcttcacgacaggtgtcaacctctcctcgaagggagacaaactgggacagcaataccagactcctgagtctgctgttggacgcggtgccgactttatcattgctggtcgtggaatttatgctgctcctgatcccgtggaggcagcgaagcggtaccagaaagagggatgggatgcataccagaagcgtgttggtgcgcaataagtagtggtgaatacgtgctctttttatggcagtatatcgcaagtatgatgcgattcataaattcagcagtcgaattctacgagagaacgatgctaagagataccctctctatatgaataatatgcctgcctcgagatatggacatattcaagatcagagttaagggtcatgtttcaaaatcacaccaatctccaacatagacgagaatttttaccggattgtctgaaggtgcagctggagattggtctattttctaagagtggggtatcactaatgtacagtcggtcactatcgtacaaacaatcacaattatatacaagatttcccatcaccccttactctaacatggcacttttatccatcgagtccgagcctagccaccatttggtgctttcgtagagaccaaagtataaccctgatccgacagcggccataaacgtgttgatagcacaccctcggaatagtcctctcgggccatctgttcgtataatctcccgtacggtattgatcatccttttcttctgaggtgcgg

SEQ ID NO: 10 - DNA, 40 bp, Artificial Sequence
  Ptef reverse primer
agatggagacttcattttgaaggtggtgcgaactttgtag

SEQ ID NO: 11 - DNA, 38 bp, Artificial Sequence
  Talp forward primer
ggtgcctcggcttgagtaccaggagtacattggagagt

SEQ ID NO: 12 - protein, 498 aa, Crotalus adamanteus
AHDRNPLEECFRETDYEEFLEIAKNGLTATSNPKRVVIVGAGMAGLSAAYVLAGAGHQVTVLEASERVGGRVRTYRKKDWYANLGPMRLPTKHRIVREYIKKFDLKLNEFSQENENAWYFIKNIRKRVREVKNNPGLLEYPVKPSEEGKSAAQLYVESLRKVVEELRSTNCKYILDKYDTYSTKEYLLKEGNLSPGAVDMIGDLLNEDSGYYVSFIESLKHDDIFGYEKRFDEIVGGMDQLPTSMYEAIKEKVQVHFNARVIEIQQNDREATVTYQTSANEMSSVTADYVIVCTTSRAARRIKFEPPLPPKKAHALRSVHYRSGTKIFLTCTKKFWEDDGIHGGKSTTDLPSRFIYYPNHNFTSGVGVIIAYGIGDDANFFQALDFKDCADIVINDLSLIHELPKEDIQTFCHPSMIQRWSLDKYAMGGITTFTPYQFQHFSEALTAPFKRIYFAGEYTAQFHGWIDSTIKSGLTAARDVNRASENPSGIHLSNDNEF

SEQ ID NO: 13 - protein, 498 aa, Crotalus atrox; identical to SEQ ID NO: 12

SEQ ID NO: 14 - protein, 618 aa, Trichoderma viride
MDNVDFAESVRTRWARRLIREKVAKELNILTERLGEVPGIPPPREGRFLGGGYSHDNLPSDPLYSSIKPALLKEAPRAEEELPPRKVCIVGAGVSGLYIAMILDDLKIPNLTYDIFESSSRTGGRLYTHHFTDAKHDYYDIGAMRYPDIPSMKRTFNLFKRTGMPLIKYYLDGENTPQLYNNHFFAKGVVDPYMVSVANGGTVPDDVVDSVGEKLQQAFGYYKEKLAEDFDKGFDELMLVDDMTTREYLKRGGPKGEAPKYDFFAIQWMETQNTGTNLFDQAFSESVIDSFDFDNPTKPEWYCIEGGTSLLVDAMKETLVHKVQNNKRVEAISIDLDAPDDGNMSVKIGGKDYSGYSTVFNTTALGCLDRMDLRGLNLHPTQADAIRCLHYDNSTKVALKFSYPWWIKDCGITCGGAASTDLPLRTCVYPSYNLGDTGEAVLLASYTWSQDATRIGSLVKDAPPQPPKEDELVELILQNLARLHAEHMTYEKIKEAYTGVYHAYCWANDPNVGGAFALFGPGQFSNLYPYLMRPAAGGKFHIVGEASSVHHAWIIGSLESAYTAVYQFLYKYKMWDYLRLLLERWQYGLQELETGKHGTAHLQFILGSLPKEYQVKIV

SEQ ID NO: 15 - protein, 517 aa, Aplysia californica
DGVCRNRRQCNKEVCGSSYDVAIVGAGPGGANSAYMLRESGLDIAVFEYSDRVGGRLFTYQLPNTPDVNLEIGGMRFIEGAMHRLWKVISELGLTPKVFKEGFGKEGRQRFYLRGQSLTKKQVKSGDVPYDLSPEEKANQGRLVEYYLEKLTGLQLNGGPLKREVALKLTVPDGRFLYDLTFDEALDLVASPEGKEFARDTHVFTSEVTLDASAISIFDDHLGEDYYGSEIYTLEEGMSSVPQGLLQTFLDAAKSNEFFPNNHLKALRRRTNGQYVLYFEPTTSKDGQTTINYLEPLKVVCAQRVILAMPVYALRQLDWSQLRNDRATQAYRAVRPMPASKVFMTFDQPWWLQNERKSWVTKSDALFSQMYDWQKSEASGDYILIASYADGLKAQYLRELKNQGEDIPGSDPGYNQVTVPLKDAILEHLTEAYGVERDSIPEPVTAASQFWTDYPFGCGWITWRAGYHFDDVISTMRRPSLKDEVYVVGSDYSWGLISSWIEGALETSENVINDYFL

SEQ ID NO: 16 - DNA, 69 bp, Aspergillus oryzae
  AoCDHss
atgaaacttgtcaaccgtctgctggcttcgttcttgtccgcatccaccgttctccagtcgtgctgggct

SEQ ID NO: 17 - DNA, 1554 bp, Aplysia californica
gacggcgtctgcagaaacaggcgtcagtgtaacaaagaggtgtgcggttcctcttatgacgtggccatcgtgggggcggggccagggggagctaactccgcctacatgctgagggagtctggcctggacatcgctgtgttcgagtactctgaccgggtgggtggccgtctgttcacctaccagctgcccaacacacccgacgtgaacctggagattggcggcatgaggttcatcgagggcgccatgcacagactctggaaagtcatttcagaactcggactgacccccaaggtgttcaaggaaggtttcggcaaggagggcagacagaggttctacctacgtggacagagcctgaccaagaaacaggtcaagagcggggacgtaccctatgacctcagcccggaggagaaggccaaccagggacgtctggtcgaatactacctggagaaactgaccggattacagctcaacggcggaccactcaaacgggaggtggcgctcaaactgaccgtgccggacggcagattcctctatgacctcacgtttgacgaagccctggacctggtggcctcccctgagggcaaagagttcgcccgagacacgcacgtgtttacctcagaggtcacactggacgcgtcggctatctccatcttcgatgaccacctgggagaggactactatggcagtgagatctacaccctggaggaaggaatgtcttctgtaccacaaggactgctacagacttttctggacgccgctaaatccaacgagttcttccccaacaaccacctgaaggctctgagacggaggaccaatggtcagtatgtcctgtactttgagcccaccacgtccaaggatggacaaaccaccatcaactacctggaacccctgaaggttgtgtgtgctcagagagtcatcctggccatgccggtctacgctctcagacaactggactggtcacaactcagaaatgaccgcgccacccaagcgtacagagccgtgcgtcctatgcctgccagtaaagtctttatgacctttgaccagccctggtggttgcagaatgagaggaaatcttgggtaaccaaatcagacgcgcttttcagccaaatgtatgactggcagaagtctgaggcgtctggagattacatcctgatcgccagctacgccgacggcctcaaagcccagtacttgcgggagctgaagaaccagggagaggacatcccaggctctgaccccggctacaaccaggtcaccgtacccctcaaggacgccattcttgaacacctcaccgaggcttacggcgtggagcgagactcgatcccagaacccgtgactgctgcctcccagttttggacagactacccgtttggctgcggctggatcacctggagggccggttaccattttgatgacgtcatcagcaccatgcgtcgcccgtcactgaaagatgaagtctacgtggtaggatcggattactcctggggacttatctcctcctggatagagggcgctcttgagacatcggaaaacgtcatcaacgactacttcctctag

SEQ ID NO: 18 - DNA, 1554 bp, Aplysia californica
gatggggtttgtcggaatcgccgccaatgcaacaaggaggtttgcggttcttcctacgacgtggctatcgtgggagccggccctggcggcgctaactctgcttacatgctccgcgaatctgggttggacatagctgtattcgaatattccgaccgagtcggcggtcgactcttcacctatcagctccctaacacccccgacgtcaatctcgaaattggaggtatgcgcttcatcgaaggggccatgcaccgtctttggaaagtaatatccgaactcggccttacgcctaaggtcttcaaggaggggttcggtaaggaaggtcgacagcgtttttatctgcgcggccaatcgctcaccaaaaagcaagtgaaaagtggtgatgtgccctacgacctgtctcctgaggaaaaggccaatcagggccgcctggtcgagtactatctggagaagctcaccggactccagctcaatggcggtcccctcaaacgagaagtggcgctgaagctcaccgtccccgatggccgttttctctatgatctcaccttcgacgaggctcttgacctggtcgcctccccagagggtaaagaattcgcacgcgatacccacgttttcaccagcgaagtcactctggacgcatctgccatcagtattttcgacgaccatcttggtgaggattattatggcagcgaaatctataccttggaggaaggcatgtcctccgtcccccaaggccttctgcaaactttccttgatgccgccaagagcaacgaatttttccccaataatcacctgaaggcccttcgacggcggacgaacggccagtacgtcttgtattttgagcctactacgtctaaggacggacaaacaacgatcaattacctggagcctctgaaggttgtctgcgcgcaacgggtgatcctggctatgccagtatacgccctgcgtcaattggattggtctcagctacgtaatgatcgggccactcaagcatatagggcagtcaggcctatgccggcttcaaaagtatttatgacgttcgatcagccgtggtggcttcagaacgagaggaagagctgggtgactaagagtgatgcactatttagtcagatgtacgattggcagaaaagcgaagcgagtggtgactacattcttattgcttcgtatgcggacggtcttaaagctcaatacttgagagagctaaaaaaccagggagaggatatcccggggagcgatccggggtacaaccaggtgacagtaccattgaaggacgcgatcctagagcatttgactgaagcatacggtgtggaaagagacagcattccggagccagttactgcggcatcgcagttctggacagactatccatttggatgtgggtggatcacatggagagcaggataccattttgatgatgtgatttcgacaatgaggagaccatcattgaaggatgaggtttacgttgttggatcagattactcgtggggactaatctcatcatggattgagggagcactagagacatcggagaacgttattaacgattattttttgtag

SEQ ID NO: 19 - protein, 498 aa, Crotalus adamanteus; identical to SEQ ID NO: 12

SEQ ID NO: 20 - DNA, 12 bp, Artificial Sequence
  Synthetic gene (partial)
accaccttcaaa

SEQ ID NO: 21 - DNA, 12 bp, Artificial sequence
  Synthetic gene (partial)
gtaccaggagta

SEQ ID NO: 22 - DNA, 28 bp, Artificial sequence
  reverse primer
cattttgaaggtggtgcgaactttgtag

SEQ ID NO: 23 - DNA, 28 bp, Artificial sequence
  forward primer
tgagtaccaggagtacattggagagttc

(19) United States
(12) Patent Application Publication
(10) Pub. No.: US 2022/0396822 A1
(43) Pub. Date: Dec. 15, 2022

(54) Method for Measuring Pentosidine and Measurement Kit

(71) Applicant: Kikkoman Corporation, Chiba (JP)

(72) Inventors: Kazuya Marushima, Helios (SG); Yi Hsin Chen, Helios (SG); Dian Kartikasari Lianto, Helios (SG); Takuya Sato, Helios (SG); Atsushi Ichiyanagi, Chiba (JP); Yasuko Araki, Chiba (JP)

(21) Appl. No.: 17/638,837

(22) PCT Filed: Aug. 21, 2020
(86) PCT No.: PCT/JP2020/031553

§ 371 Date: Feb. 27, 2022

(30) Foreign Application Priority Data
Aug. 27, 2019 (JP) 2019-154965

(51) Int. Cl. C12Q 1/26; C12N 9/04

(52) U.S. Cl. CPC C12Q 1/26; C12N 9/0006

(57) ABSTRACT

Provided is a method for measuring pentosidine in a specimen, the measurement method comprising the steps of: degrading the specimen with an amino acid degrading enzyme; contacting the specimen after the degradation step with a protein having activity that oxidatively degrades pentosidine; and detecting change resulting from the contact, wherein the amino acid degrading enzyme and the protein having activity that oxidatively degrades pentosidine are different from each other.

### TECHNICAL FIELD

The present invention relates to a method for measuring pentosidine, etc. More specifically, the present invention relates to a method for measuring pentosidine, etc., which reduces a measurement error to obtain an accurate measurement value.

### BACKGROUND ART

Pentosidine ((2S)-2-amino-6-[2-[[(4S)-4-amino-4-carboxybutyl]amino]imidazo[4,5-b]pyridin-4-yl]hexanoic acid) has a structure where pentose, and equimolar lysine and arginine are cross-linked, and is known to accumulate in the human skin in correlation with aging or the development of diabetes mellitus and to increase, particularly, during the development of diabetes mellitus or in end-stage nephropathy.

It is known that pentosidine can be quantified by HPLC with its fluorescence (Ex: 335 nm, Em: 385 nm) as an index after acid hydrolysis, and can also be quantified by use of an immunochemical method (e.g., ELISA) using a monoclonal antibody against pentosidine.

Pentosidine is known to be associated with schizophrenia in addition to aging or diabetes mellitus. For example, a method for testing schizophrenia, comprising the step of measuring an amount of pentosidine with the intended use for biological samples is disclosed (see e.g., Patent Literature 1).

### CITATION LIST

### Patent Literature

Patent Literature 1: Japanese Patent No. 5738346

### SUMMARY OF INVENTION

### Technical Problem

The quantification of pentosidine by an immunochemical method or an instrumental analytical approach may be complicated and expensive. An object of the present invention is to provide an inexpensive and simple method for measuring pentosidine using a novel enzyme, as compared with an immunochemical method or an instrumental analytical approach. Particularly, an object of the present invention is to provide a method for measuring pentosidine which reduces a measurement error to obtain an accurate measurement value.

### Solution to Problem

The present inventors have completed the present invention by finding that a novel enzyme identified from a filamentous fungus is useful in the quantification of pentosidine and finding that use of this enzyme in combination with an amino acid degrading enzyme more reduces a measurement error.

The present invention is summarized as follows.

[1]

A method for measuring pentosidine in a specimen, the measurement method comprising the steps of:

degrading the specimen with an amino acid degrading enzyme;

contacting the specimen after the degradation step with a protein having activity that oxidatively degrades pentosidine; and

detecting change resulting from the contact, wherein the amino acid degrading enzyme and the protein having activity that oxidatively degrades pentosidine are different from each other.

[2]

The measurement method according to [1], wherein in the detection step, change in an amount of oxygen, hydrogen peroxide or ammonia is detected.

[3]

The measurement method according to [1] or [2], wherein the protein having activity that oxidatively degrades pentosidine has the following physicochemical properties:

(1) action: activity that oxidatively degrades pentosidine; and
(2) molecular weight based on SDS-PAGE: 75,000 to 85,000.
[4]

The measurement method according to any of [1] to [3], wherein the protein having activity that oxidatively degrades pentosidine is any protein selected from the group consisting of the following (a) to (f):

(a) a protein consisting of the amino acid sequence as set forth in SEQ ID NO: 2 or SEQ ID NO: 4;
(b) a protein encoded by a gene consisting of the nucleotide sequence as set forth in SEQ ID NO: 1, SEQ ID NO: 3, SEQ ID NO: 5 or SEQ ID NO: 6;
(c) a protein consisting of an amino acid sequence having 75% or higher identity to the amino acid sequence as set forth in SEQ ID NO: 2 or SEQ ID NO: 4;
(d) a protein encoded by a gene consisting of a nucleotide sequence having 75% or higher identity to the nucleotide sequence as set forth in SEQ ID NO: 1, SEQ ID NO: 3, SEQ ID NO: 5 or SEQ ID NO: 6;
(e) a protein consisting of an amino acid sequence derived from the amino acid sequence as set forth in SEQ ID NO: 2 or SEQ ID NO: 4 by the deletion, substitution and/or addition of one or more amino acids; and
(f) a protein encoded by a nucleotide sequence that hybridizes under stringent conditions to the nucleotide sequence as set forth in SEQ ID NO: 1, SEQ ID NO: 3, SEQ ID NO: 5 or SEQ ID NO: 6.
[5]

The measurement method according to any of [1] to [4], wherein the protein having activity that oxidatively degrades pentosidine is derived from a filamentous fungus.

[6]

The measurement method according to any of [1] to [5], wherein

the protein having activity that oxidatively degrades pentosidine is pentosidine oxidase, and

the amino acid degrading enzyme degrades an amino acid contained in the specimen, wherein the amino acid is selected from arginine, leucine, methionine, phenylalanine, tryptophan and tyrosine.

[6′]

The measurement method according to any of [1] to [5], wherein the amino acid degrading enzyme degrades an amino acid selected from arginine, leucine, methionine, phenylalanine, tryptophan and tyrosine.

[7]

The measurement method according to any of [1] to [6], wherein the amino acid to be degraded by the amino acid degrading enzyme is an amino acid against which the protein having activity that oxidatively degrades pentosidine has 40% or higher relative activity when the activity of the protein having activity that oxidatively degrades pentosidine against pentosidine is defined as 100%.

[8]

The measurement method according to any of [1] to [7], wherein the amino acid degrading enzyme is selected from the group consisting of amino acid oxidase, amino acid dehydrogenase, amino acid aminotransferase, amino acid decarboxylase, amino acid ammonia lyase, amino acid oxygenase and amino acid hydrolase.

[9]

A kit for measuring pentosidine in a specimen, comprising:

(i) an amino acid degrading enzyme; and
(ii) a protein having activity that oxidatively degrades pentosidine.
[10]

The kit according to [9], wherein the protein having activity that oxidatively degrades pentosidine is any protein selected from the group consisting of the following (a) to (f):

(a) a protein consisting of the amino acid sequence as set forth in SEQ ID NO: 2 or SEQ ID NO: 4;
(b) a protein encoded by a gene consisting of the nucleotide sequence as set forth in SEQ ID NO: 1, SEQ ID NO: 3, SEQ ID NO: 5 or SEQ ID NO: 6;
(c) a protein consisting of an amino acid sequence having 75% or higher identity to the amino acid sequence as set forth in SEQ ID NO: 2 or SEQ ID NO: 4;
(d) a protein encoded by a gene consisting of a nucleotide sequence having 75% or higher identity to the nucleotide sequence as set forth in SEQ ID NO: 1, SEQ ID NO: 3, SEQ ID NO: 5 or SEQ ID NO: 6;
(e) a protein consisting of an amino acid sequence derived from the amino acid sequence as set forth in SEQ ID NO: 2 or SEQ ID NO: 4 by the deletion, substitution and/or addition of one or more amino acids; and
(f) a protein encoded by a nucleotide sequence that hybridizes under stringent conditions to the nucleotide sequence as set forth in SEQ ID NO: 1, SEQ ID NO: 3, SEQ ID NO: 5 or SEQ ID NO: 6.

The kit according to [9] or [10], wherein the amino acid degrading enzyme is an enzyme that degrades an amino acid selected from arginine, leucine, methionine, phenylalanine, tryptophan and tyrosine.

[12]

A method for producing a reaction product of pentosidine derived from a specimen, the method comprising the steps of:

degrading the specimen with an amino acid degrading enzyme; and

contacting the specimen after the degradation step with a protein having activity that oxidatively degrades pentosidine, wherein

the amino acid degrading enzyme and the protein having activity that oxidatively degrades pentosidine are different from each other.
[13]

The measurement method according to any of [1] to [8], wherein the amino acid degrading enzyme comprises Aplysia californica-derived escapin and/or Crotalus adamanteus-derived amino acid oxidase.

[14]

The measurement method according to any of [1] to [8], wherein the amino acid degrading enzyme degrades an amino acid contained in the specimen, wherein the amino acid is selected from asparagine, glutamine and histidine.

[14′]

The measurement method according to any of [1] to [7], wherein the amino acid degrading enzyme degrades an amino acid selected from asparagine, glutamine and histidine.

[15]

The kit according to any of [9] to [11], further comprising (iii) at least one member selected from a reagent for hydrogen peroxide detection, a reagent for ammonia detection, a reagent for pentosidine deamination product detection and a reagent for oxygen detection.

### Advantageous Effects of Invention

The present invention enables pentosidine to be conveniently and rapidly detected and quantified by an enzymatic method. In this respect, a measurement error is reduced to obtain an accurate measurement value.

### BRIEF DESCRIPTION OF DRAWINGS

FIG. 1 shows results of a substrate concentration dependence test on a partially purified enzyme liquid (Elution 1) fractionated by use of anion-exchange chromatography from Sarocladium sp. ΔOD (ordinate) was plotted against a final pentosidine concentration (abscissa). Data after 20 minutes from the start of reaction was used.

FIG. 2 shows results of a thermal deactivation test on the partially purified enzyme liquid (Elution 1). The results were of analyzing the deactivation of enzymatic activity by heat treatment and corresponded to data after 20 minutes from the start of reaction.

FIG. 3 shows results of measuring the concentration of hydrogen peroxide produced through the reaction of pentosidine with pentosidine oxidase. The concentration of hydrogen peroxide was measured at absorbance of 658 nm.

FIG. 4 shows the relationship between the final concentration of pentosidine and the amount of elevation in A₆₅₈ (ΔA) caused by the oxidation of pentosidine.

FIG. 5 shows a putative mechanism of reaction through which pentosidine oxidase degrades pentosidine. The drawing illustrates a manner in which the respective amino groups of lysine and arginine constituting pentosidine are oxidatively deaminated to form hydrogen peroxide and ammonia.

FIG. 6A shows the sequences of SEQ ID NO: 1 and SEQ ID NO: 2.

FIG. 6B shows the sequences of SEQ ID NO: 3 and SEQ ID NO: 4.

FIG. 6C shows the sequences of SEQ ID NO: 5 and SEQ ID NO: 6.

FIG. 6D shows the sequences of SEQ ID NO: 7 to SEQ ID NO: 11.

FIG. 6E shows the sequences of SEQ ID NO: 12 to SEQ ID NO: 14.

FIG. 7 shows the range of the optimum pH of PenOX2.

FIG. 8 shows the range of the optimum temperature of PenOX2.

FIG. 9 shows the range of the heat stability of PenOX2.

FIG. 10 shows the range of the stable pH of PenOX2.

FIG. 11 shows the Km value of PenOX2 for pentosidine.

FIG. 12 shows the molecular weight of PenOX2.

FIG. 13 shows the substrate specificity of amino acid degrading enzyme 1 measured in Example 13.

FIG. 14 shows the substrate specificity of amino acid degrading enzyme 2 measured in Example 14.

FIG. 15 shows the substrate specificity of pentosidine oxidase measured in Example 15.

### DESCRIPTION OF EMBODIMENTS

Hereinafter, the method for measuring pentosidine, etc. according to one aspect of the present invention (hereinafter, also referred to as the “present embodiment”) will be described in detail. However, the technical scope of the present invention is not limited by the items of the present embodiment. The present invention can assume various forms as long as the object of the present invention is attained.

(Protein Having Activity that Oxidatively Degrades Pentosidine)

In one aspect, the present embodiment relates to a method for measuring pentosidine in a specimen.

Pentosidine has a structure where pentose, and equimolar lysine and arginine are cross-linked, as described above. The “protein having activity that oxidatively degrades pentosidine” for use in the measurement method of the present embodiment is not limited as long as the protein has such degrading activity. The protein includes pentosidine oxidase having pentosidine oxidase activity and pentosidine dehydrogenase having pentosidine dehydrogenase activity.

The protein having activity that oxidatively degrades pentosidine described in Examples mentioned later is a novel enzyme and has at least pentosidine oxidase activity. As used herein, the “pentosidine oxidase activity” means activity that oxidatively degrades pentosidine, more specifically, activity that oxidizes pentosidine to produce its deamination product, hydrogen peroxide, and ammonia, or activity that consumes oxygen.

As used herein, the “pentosidine dehydrogenase activity” means activity that oxidatively degrades pentosidine, more specifically, activity that oxidizes pentosidine to produce its deamination product, a reduced coenzyme, and ammonia, or activity that consumes an oxidized coenzyme. In this context, examples of the coenzyme include flavin adenine dinucleotide (FAD), flavin mononucleotide (FMN), nicotinamide adenine dinucleotide (NAD), and nicotinamide adenine dinucleotide phosphate (NADP). The reduced coenzyme may further reduce a mediator. The mediator is not particularly limited as long as the mediator can transfer or accept electrons to or from a coenzyme contained in the pentosidine dehydrogenase of the present invention. Examples of the mediator include, but are not limited to, quinones, phenazines, ferricyanides, osmium salts or complexes, ruthenium salts or complexes, nitrosoanilines, aminoanilines, viologens, cytochromes, phenoxazines, phenothiazines, ferredoxins, ferrocenes, and derivatives thereof. Examples of the quinones include, but are not limited to, naphthoquinone and derivatives thereof (e.g., naphthoquinone-4-sulfonate), phenanthrolinequinone and derivatives thereof, and phenanthrenequinone and derivatives thereof. Examples of the phenazines include, but are not limited to, phenazine methosulfate (PMS) and derivatives thereof (e.g., 1-methoxy PMS and 1-ethoxy PMS). Examples of the ferricyanides include, but are not limited to, potassium ferricyanide. Examples of the osmium salts or complexes include, but are not limited to, osmium chloride and hexaammineosmium. Examples of the ruthenium salts or complexes include, but are not limited to, ruthenium chloride and hexaammineruthenium. Examples of the nitrosoanilines include, but are not limited to, N,N-dimethyl-4-nitrosoaniline and N,N-bis-hydroxyethyl-4-nitrosoaniline and their derivatives. Other examples of the mediator also include mediators known to those skilled in the art. In the specification of the present application, the term “mediator” includes neither oxygen nor hydrogen peroxide unless otherwise specified.

It should be understood that every protein and a gene encoding the protein are included, without being limited by a particular sequence, in the scope of the present embodiment as long as the protein has the enzymatic activity as described above. The nucleotide sequence and the amino acid sequence of the pentosidine oxidase as the protein having activity that oxidatively degrades pentosidine will be described below by taking an enzyme derived from a filamentous fungus of the genus Sarocladium as an example.

(Amino Acid Sequence of Pentosidine Oxidase)

The pentosidine oxidase is not particularly limited by its amino acid sequence as long as the pentosidine oxidase has the enzymatic activity described above. Examples of one form of the enzyme having the pentosidine oxidase activity described above include a protein having the amino acid sequence represented by SEQ ID NO: 2 or SEQ ID NO: 4. Hereinafter, the proteins having the amino acid sequences represented by SEQ ID NO: 2 and SEQ ID NO: 4 are also referred to as pentosidine oxidase 1 (or PenOX1) and pentosidine oxidase 2 (or PenOX2), respectively. A gene (g4462) encoding the pentosidine oxidase 1 is presumably constituted by six exons and five introns, whereas a gene (g10122) encoding the pentosidine oxidase 2 is presumably constituted by two exons and one intron.

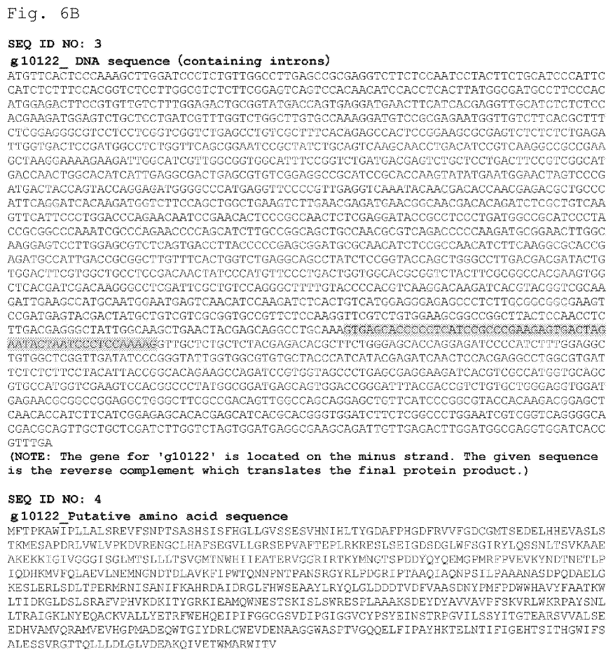

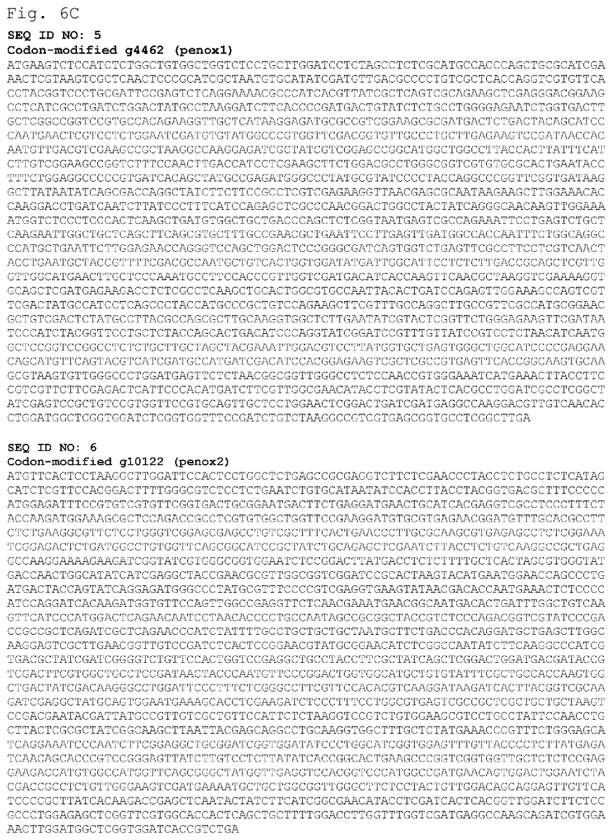

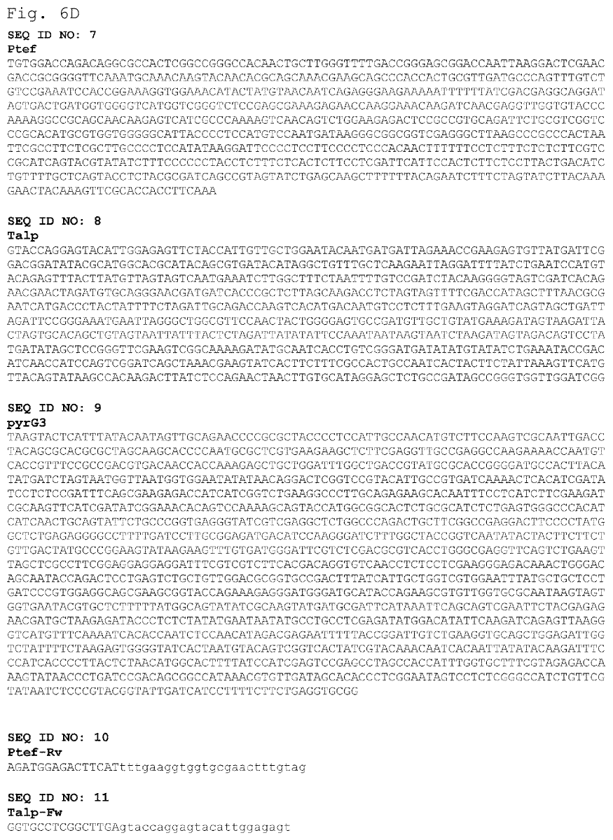

The pentosidine oxidase having the amino acid sequence represented by SEQ ID NO: 2 or SEQ ID NO: 4 is derived from a filamentous fungus of the genus Sarocladium. The nucleotide sequences of the genes encoding these enzymes are the nucleotide sequences represented by SEQ ID NO: 1 and SEQ ID NO: 3, respectively. FIG. 6 shows the amino acid sequences and the nucleotide sequences of the enzymes.

The amino acid sequence of the pentosidine oxidase may consist of an amino acid sequence derived from the amino acid sequence of the wild-type enzyme, such as SEQ ID NO: 2 or SEQ ID NO: 4, by the deletion, substitution, addition, or the like of one or more amino acids, for example, 1, 2, 3, 4, 5, 6, 7, 8, 9, 10, 11, 12, 13, 14, 15, 16, 17, 18, 19, 20, 21, 22, 23, 24 or 25, preferably several amino acids, per unit (one unit involves 100 amino acids in the amino acid sequence) as long as the resulting pentosidine oxidase has the enzymatic activity of the pentosidine oxidase described above. In this context, the range of “one to several” in the “deletion, substitution, or addition of one to several amino acids” in the amino acid sequence is not particularly limited and means preferably approximately 1, 2, 3, 4, 5, 6, 7, 8, 9 or 10, more preferably approximately 1, 2, 3, 4 or 5, per unit described above. The “deletion of an amino acid” means the elimination or disappearance of an amino acid residue in a sequence. The “substitution of an amino acid” means the replacement of an amino acid residue in a sequence with another amino acid residue. The “addition of an amino acid” means the insertion of an additional amino acid residue into a sequence.

A specific form of the “deletion, substitution, or addition of an amino acid” is a form in which an amino acid is replaced with another amino acid chemically similar thereto to an extent that the pentosidine oxidase activity is maintained. Examples thereof can include the case of substituting a hydrophobic amino acid with another hydrophobic amino acid and the case of substituting a polar amino acid with another polar amino acid having the same charge thereas. Such chemically similar amino acids are known in the art on an amino acid basis.

Specifically, examples of the nonpolar (hydrophobic) amino acid include alanine, valine, isoleucine, leucine, proline, tryptophan, phenylalanine, and methionine. Examples of the polar (neutral) amino acid include glycine, serine, threonine, tyrosine, glutamine, asparagine, and cysteine. Examples of the basic amino acid having positive charge include arginine, histidine, and lysine. Examples of the acidic amino acid having negative charge include aspartic acid and glutamic acid.

Examples of the amino acid sequence of the pentosidine oxidase also include an amino acid sequence having sequence identity above a certain level to the amino acid sequence of the wild-type enzyme, such as SEQ ID NO: 2 or SEQ ID NO: 4, and include an amino acid sequence having 75% or higher, preferably 80% or higher, more preferably 85% or higher, more preferably 90% or higher, most preferably 95% or higher identity, to the amino acid sequence of the pentosidine oxidase.

(Gene Encoding Pentosidine Oxidase)

The gene encoding the pentosidine oxidase (hereinafter, also referred to as the “pentosidine oxidase gene”) is not particularly limited as long as the gene comprises a nucleotide sequence encoding the amino acid sequence of the enzyme having the pentosidine oxidase activity described above. In some aspects, the pentosidine oxidase gene is expressed in a transformant which thereby produces pentosidine oxidase.

As used herein, the “expression of a gene” means that an enzyme encoded by the gene is produced in a form having original catalytic activity via transcription, translation, etc. The “expression of a gene” also encompasses the high expression of the gene, i.e., the production of an enzyme encoded by the gene in an amount exceeding an original expression level from a host organism by the insertion of the gene.

The pentosidine oxidase gene may be a gene capable of producing the pentosidine oxidase via splicing after transcription of the gene or may be a gene capable of producing the pentosidine oxidase without the mediation of splicing after transcription of the gene, when transferred to a host organism.

The pentosidine oxidase gene may not be completely identical to a gene originally carried (i.e., a wild-type gene) by a source organism such as a filamentous fungus of the genus Sarocladium, and may be DNA having a nucleotide sequence that hybridizes under stringent conditions to a nucleotide sequence complementary to the nucleotide sequence of the wild-type gene as long as the gene encodes the enzyme having the pentosidine oxidase activity described above.

As used herein, the “nucleotide sequence that hybridizes under stringent conditions” means the nucleotide sequence of DNA that is obtained by use of colony hybridization, plaque hybridization, Southern blot hybridization, or the like using, as a probe, DNA corresponding to a portion of the nucleotide sequence of the wild-type gene, such as SEQ ID NO: 1 or SEQ ID NO: 3.

As used herein, the “stringent conditions” are conditions under which the signal of a specific hybrid is clearly discriminated from the signal of a nonspecific hybrid, and differ depending on the hybridization system used and the type, sequence and length of the probe. Such conditions can be determined by changing a hybridization temperature or changing a washing temperature and a salt concentration.

For example, if the signal of a nonspecific hybrid is strongly detected, the specificity can be enhanced by elevating the hybridization and washing temperatures while lowering, if necessary, the salt concentration for washing. If even the signal of a specific hybrid is not detected, the hybrid can be stabilized by lowering the hybridization and washing temperatures while elevating, if necessary, the salt concentration for washing.

In some aspects, specific examples of the stringent conditions include the following: a DNA probe is used as the probe, and the hybridization is performed overnight (approximately 8 to 16 hours) using 5×SSC, 1.0% (w/v) blocking reagent for nucleic acid hybridization (manufactured by Boehringer Mannheim GmbH), 0.1% (w/v) N-lauroylsarcosine, and 0.02% (w/v) SDS. The washing is performed twice for 15 minutes each using 0.1 to 0.5×SSC and 0.1% (w/v) SDS, preferably 0.1×SSC and 0.1% (w/v) SDS. The temperatures at which the hybridization and the washing are performed are 65° C. or higher, preferably 68° C. or higher.

Examples of the DNA having a nucleotide sequence that hybridizes under stringent conditions can include DNA having the nucleotide sequence of the wild-type gene derived from a colony or a plaque, DNA that is obtained by hybridization under the stringent conditions described above using a filter on which a fragment of the DNA is immobilized, and DNA that can be identified by carrying out hybridization at 40 to 75° C. in the presence of 0.5 to 2.0 M NaCl, then carrying out hybridization at 65° C., preferably in the presence of 0.7 to 1.0 M NaCl, and then washing the filter under a condition of 65° C. using a 0.1 to 1×SSC solution (the 1×SSC solution consists of 150 mM sodium chloride and 15 mM sodium citrate). Methods for probe preparation or hybridization can be carried out in accordance with methods described in Molecular Cloning: A laboratory Manual, 2nd-Ed., Cold Spring Harbor Laboratory, Cold Spring Harbor, N.Y., 1989, and Current Protocols in Molecular Biology, Supplement 1-38, John Wiley & Sons, 1987-1997 (hereinafter, these literatures are also referred to as “technical references”).

Those skilled in the art can appropriately set conditions for obtaining the DNA having a nucleotide sequence that hybridizes under stringent conditions to a nucleotide sequence complementary to the nucleotide sequence of the wild-type gene, by taking into consideration such conditions such as salt concentrations of buffers and temperatures as well as other conditions such as a probe concentration, a probe length, and a reaction time.

Examples of the DNA comprising a nucleotide sequence that hybridizes under stringent conditions include DNA having sequence identity above a certain level to the nucleotide sequence of DNA having the nucleotide sequence of the wild-type gene for use as a probe, and include DNA having 75% or higher, preferably 80% or higher, more preferably 85% or higher, more preferably 90% or higher, further preferably 95% or higher identity, to the nucleotide sequence of the wild-type gene.

The nucleotide sequence that hybridizes under stringent conditions to a nucleotide sequence complementary to the nucleotide sequence of the wild-type gene includes, for example, a nucleotide sequence derived from the nucleotide sequence of the wild-type gene by the deletion, substitution, addition, or the like of one or more bases, for example, 1 to 125, 1 to 100, 1 to 75, 1 to 50, 1 to 30 or 1 to 20, preferably 1 to several, for example, 1, 2, 3, 4, 5, 6, 7, 8, 9 or 10 bases, per unit (one unit involves 500 bases in the nucleotide sequence).

In this context, the “deletion of a base” means the elimination or disappearance of a base in a sequence. The “substitution of a base” means the replacement of a base in a sequence with another base. The “addition of a base” means the insertion of an additional base.

An enzyme encoded by the nucleotide sequence that hybridizes under stringent conditions to a nucleotide sequence complementary to the nucleotide sequence of the wild-type gene has the probability of being an enzyme having an amino acid sequence derived from the amino acid sequence of the enzyme encoded by the nucleotide sequence of the wild-type gene, by the deletion, substitution, addition, or the like of one or more, preferably several amino acids, but has the same enzymatic activity as that of the enzyme encoded by the nucleotide sequence of the wild-type gene.

The gene encoding the enzyme is a nucleotide sequence encoding an amino acid sequence identical or analogous to the amino acid sequence of the enzyme encoded by the wild-type gene, and may comprise a nucleotide sequence different from that of the wild-type gene, by exploiting several types of codons corresponding to one amino acid. Examples of such a codon-modified nucleotide sequence of the nucleotide sequence of the wild-type gene include the nucleotide sequence as set forth in SEQ ID NO: 5 (penox1) (codon-modified form of g4462) and SEQ ID NO: 6 (penox2) (codon-modified form of g10122) (FIG. 6C). The codon-modified nucleotide sequence is preferably, for example, a nucleotide sequence that has undergone codon modification so as to facilitate expression in a host organism.

(Approach for Calculating Sequence Identity)

The method for determining the sequence identity of a nucleotide sequence or an amino acid sequence is not particularly limited. The sequence identity is determined, for example, by using a program for aligning the wild-type gene or the amino acid sequence of the enzyme encoded by the wild-type gene with a targeted nucleotide sequence or amino acid sequence, and calculating the percent match between the sequences, usually by use of a known method.

For example, the algorithm of Karlin and Altschul (Proc. Natl. Acad. Sci. USA 87: 2264-2268, 1990; and Proc. Natl. Acad. Sci. USA 90: 5873-5877, 1993) is known as a program for calculating the percent match between two amino acid sequences or nucleotide sequences, and BLAST program using this algorithm has been developed by Altschul et al. (J. Mol. Biol. 215: 403-410, 1990). Further, gapped BLAST is also known as a program for determining sequence identity with higher sensitivity than that of BLAST (Nucleic Acids Res. 25: 3389-3402, 1997). Thus, those skilled in the art can utilize, for example, any of the programs described above to search a database for a sequence that exhibits high sequence identity to a given sequence. These are available in the internet website of the National Center for Biotechnology Information, U.S. (http://blast.ncbi.nlm.nih.gov/Blast.cgi).

Each of the methods described above can usually be used for searching a database for a sequence that exhibits sequence identity. Homology analysis of Genetyx network version 12.0.1 (manufactured by Genetyx Corp.) may be used as an approach of determining the sequence identity of an individual sequence. This method is based on the Lipman-Pearson method (Science 227: 1435-1441, 1985). A region (CDS or ORF) encoding a protein is used, if possible, in analyzing the sequence identity of a nucleotide sequence.

(Origin of Gene Encoding Enzyme)

The gene encoding the enzyme is derived from an organism species having the ability to produce pentosidine oxidase. Examples of the source organism of the gene encoding the enzyme include microbes such as filamentous fungi. Specific examples of the microbe having the ability to produce pentosidine oxidase include the genus Sarocladium.

As described above, the source organism of the gene encoding the enzyme is not particularly limited, and an enzyme expressed in a transformant preferably exhibits activity without being inactivated under growing conditions of a host organism. Accordingly, the source organism of the gene encoding the enzyme is preferably a microbe similar in growing conditions to the host organism to be transformed by the insertion of the gene encoding the enzyme.

Examples of the distinctive physicochemical characteristics or properties of the enzyme having the pentosidine oxidase activity include the following:

Molecular weight based on SDS-PAGE: 75,000 to 85,000

Optimum pH: pH of approximately 6.5 to 8.0

The optimum pH is pH at which the enzyme acts most suitably, and the pentosidine oxidase is also capable of acting at pH other than the range described above.

Optimum temperature: Approximately 37 to 50° C.

The optimum temperature is a temperature at which the enzyme acts most suitably, and the pentosidine oxidase is also capable of acting at temperature other than the temperature range described above.

Temperature stability: 90% or more of the pentosidine oxidase activity is maintained when the enzyme is preserved at 30° C. for 10 minutes. 50% or more of the pentosidine oxidase activity is maintained when the enzyme is preserved at 40° C. for 10 minutes.

pH stability: 60% or more of the pentosidine oxidase activity is maintained in the range of pH 4.0 to 9.0.

Km value: The Km value for pentosidine is 1 mM or less.

The Km value is a Michaelis constant. A specific calculation method therefor is not particularly limited, and the Km value can be calculated by an arbitrarily selected known method. The Km value can be calculated, for example, according to an Michaelis-Menten equation drawn by a method based on the Lineweaver-Burk plot, as in a method described in Example 9 mentioned later.

(Cloning of Gene Encoding Enzyme by Genetic Engineering Approach)

The gene encoding the enzyme can be inserted into any of appropriate various known vectors. Further, this vector can be transferred to an appropriate known host organism to prepare a transformant harboring a recombinant vector (recombinant DNA) containing the gene encoding the enzyme. Those skilled in the art can appropriately select a method for obtaining the gene encoding the enzyme, a method for obtaining information on the nucleotide sequence of the gene encoding the enzyme and the amino acid sequence of the enzyme, a method for producing various vectors, a method for preparing the transformant, etc. As used herein, the transformation and the transformant encompass transduction and a transductant, respectively. One non-limiting example of the cloning of the gene encoding the enzyme will be mentioned later.

A usual gene cloning method generally used can be appropriately used for cloning the gene encoding the enzyme. For example, chromosomal DNA or mRNA can be extracted from a microbe or various cells having the ability to produce the enzyme by a routine method, for example, a method described in the technical references (supra). cDNA can be synthesized with the extracted mRNA as a template. A chromosomal DNA or cDNA library can be prepared using the chromosomal DNA or the cDNA thus obtained.

In some aspects, the gene encoding the enzyme can be obtained by cloning with chromosomal DNA or cDNA of a source organism having the gene as a template. Examples of the source organism of the gene encoding the enzyme can include, but are not particularly limited to, Sarocladium sp. described above. For example, Sarocladium sp. is cultured, and water is removed from the obtained fungus body, which is then physically ground into fungus body pieces in a fine powder form using a mortar or the like while cooled in liquid nitrogen. A chromosomal DNA fraction is extracted from the fungus body pieces by a usual method. A commercially available chromosomal DNA extraction kit such as DNeasy Plant Mini Kit (manufactured by Qiagen N.V.) can be used in chromosomal DNA extraction operation.

Subsequently, DNA is amplified by polymerase chain reaction (hereinafter, referred to as “PCR”) using the chromosomal DNA as a template and primers complementary to a 5′-terminal sequence and a 3′-terminal sequence. The primers are not particularly limited as long as the primers are capable of amplifying a DNA fragment containing the gene. In another method, DNA containing a fragment of the gene of interest is amplified by appropriate PCR such as 5′ RACE or 3′ RACE and such DNAs can be linked to obtain DNA containing the full-length gene of interest.

The method for obtaining the gene encoding the enzyme is not particularly limited, and the gene encoding the enzyme can be constructed by use of, for example, a chemical synthesis method, instead of a genetic engineering approach.

The nucleotide sequence of the amplification product obtained by PCR amplification or the chemically synthesized gene can be confirmed, for example, as follows: DNA to be sequenced is inserted into an appropriate vector in accordance with a usual method to prepare recombinant DNA. A commercially available kit such as TA Cloning Kit (manufactured by Invitrogen Corp.); commercially available plasmid vector DNA such as pUC19 (manufactured by Takara Bio Inc.), pUC18 (manufactured by Takara Bio Inc.), pBR322 (manufactured by Takara Bio Inc.), pBluescript SK+ (manufactured by Stratagene California), or pYES2/CT (manufactured by Invitrogen Corp.); or commercially available bacteriophage vector DNA such as XEMBL3 (manufactured by Stratagene California) can be used in cloning into the vector. In some aspects, a host organism, for example, Escherichia coli, preferably Escherichia coli JM109 strain (manufactured by Takara Bio Inc.) or Escherichia coli DH5α strain (manufactured by Takara Bio Inc.), is transformed with the recombinant DNA. The recombinant DNA contained in the obtained transformant may be purified using QIAGEN Plasmid Mini Kit (manufactured by Qiagen N.V.) or the like.

The nucleotide sequence of each gene inserted in the recombinant DNA can be determined by a dideoxy method (Methods in Enzymology, 101, 20-78, 1983) or the like. Examples of the sequence analysis apparatus for use in determining the nucleotide sequence include, but are not particularly limited to, Li-COR MODEL 4200L sequencer (manufactured by Aloka Co., Ltd.), 370DNA sequencing system (manufactured by PerkinElmer, Inc.), and CEQ2000XL DNA analysis system (manufactured by Beckman Coulter Inc.). Then, the amino acid sequence of the protein to be obtained by translation, i.e., the enzyme, can be known on the basis of the determined nucleotide sequence.

(Construction of Recombinant Vector Containing Gene Encoding Enzyme)

The recombinant vector (recombinant DNA) containing the gene encoding the enzyme can be constructed by ligating a PCR amplification product containing any gene encoding the enzyme with any of various vectors in a form that permits expression of the gene encoding the enzyme. The recombinant vector can be constructed, for example, by excising a DNA fragment containing any gene encoding the enzyme with an appropriate restriction enzyme, and ligating the DNA fragment with a plasmid cleaved with the appropriate restriction enzyme. Alternatively, the recombinant vector can be obtained by ligating a DNA fragment containing the gene having both ends added to sequences homologous to a plasmid with a DNA fragment derived from the plasmid amplified by inverse PCR, using a commercially available recombinant vector preparation kit such as In-Fusion HD Cloning Kit (manufactured by Clontech Laboratories, Inc.) or the like.

(Method for Preparing Transformant)

Examples of the method for preparing the transformant include, but are not particularly limited to, a method of inserting the gene encoding the enzyme in an expressible form into a host organism according to a routine method. In some aspects, any gene encoding the enzyme is inserted to between an expression inducible promoter and a terminator to prepare a DNA construct. Subsequently, a host organism is transformed with the DNA construct containing the gene encoding the enzyme to obtain a transformant overexpressing the gene encoding the enzyme. In the present specification, a DNA fragment consisting of expression inducible promoter-gene encoding the enzyme-terminator prepared in order to transform a host organism, and a recombinant vector containing the DNA fragment are collectively referred to as a DNA construct.

Examples of the method for inserting the gene encoding the enzyme in an expressible form into a host organism include, but are not particularly limited to: an approach of directly inserting the gene onto a chromosome of the host organism through the use of homologous recombination or nonhomologous recombination; and an approach of ligating the gene with a plasmid vector, which is then transferred into the host organism.

In the method using homologous recombination, the DNA construct can be placed between and linked to sequences homologous to an upstream region and a downstream region of a recombination site on a chromosome, and thereby inserted into the genome of the host organism. In the method using nonhomologous recombination, the DNA construct can be inserted, even without being linked to the homologous sequences, into the genome of the host organism. Examples of the high-expression promoter include, but are not particularly limited to, a promoter region of translation elongation factor TEFL gene (tef1), a promoter region of α-amylase gene (amy), a promoter region of alkaline protease gene (alp), and a promoter region of glyceraldehyde-3-phosphate dehydrogenase (gpd).

In the method using a vector, the DNA construct is integrated into a plasmid vector for use in the transformation of a host organism by a routine method, and the corresponding host organism can be transformed therewith by a routine method.

Such a suitable vector-host system is not particularly limited as long as the system is capable of producing the enzyme in the host organism. Examples thereof include a system of pUC19 and a filamentous fungus and a system of pSTA14 (Mol. Gen. Genet. 218, 99-104, 1989) and a filamentous fungus.

The DNA construct is preferably used by transfer into a chromosome of the host organism. In other methods, the DNA construct may be integrated into an autonomous replicating vector (Ozeki et al., Biosci. Biotechnol. Biochem. 59, 1133 (1995)) and thereby used without being transferred to a chromosome.

The DNA construct may contain a marker gene for rendering transformed cells selectable. Examples of the marker gene include, but are not particularly limited to: genes, such as pyrG, niaD, and adeA, which complement the auxotrophy of the host organism; and drug resistance genes against drugs such as pyrithiamine, hygromycin B, and oligomycin. Also, the DNA construct preferably contains a promoter that permits overexpression of the gene encoding the enzyme in the host organism, a terminator, and other control sequences (e.g., an enhancer and a polyadenylation sequence). Examples of the promoter include, but are not particularly limited to, appropriate expression inducible promoters and constitutive promoters, and include tef1 promoter, alp promoter, amy promoter, and gpd promoter. Examples of the terminator include, but are not particularly limited to, alp terminator, amy terminator, and tef1 terminator.

In the DNA construct, an expression control sequence of the gene encoding the enzyme is not necessarily required when the DNA fragment containing the gene encoding the enzyme for insertion contains a sequence having an expression control function. In the case of performing transformation by a cotransformation method, the DNA construct may not have a marker gene.

The DNA construct can be tagged for purification. For example, a linker sequence is appropriately connected to upstream or downstream of the gene encoding the enzyme, and six or more codons of a nucleotide sequence encoding histidine can be connected thereto so that purification using a nickel column is attained.

The DNA construct may contain a homologous sequence necessary for marker recycling. For example, pyrG marker can be eliminated in a medium containing 5-fluoroorotic acid (5FOA) by adding a sequence homologous to a sequence upstream of an insertion site (5′ homologous recombination region) to downstream of the pyrG marker, or adding a sequence homologous to a sequence downstream of an insertion site (3′ homologous recombination region) to upstream of the pyrG marker. The length of the homologous sequence suitable for marker recycling is preferably 0.5 kb or larger.

One form of the DNA construct is, for example, a DNA construct containing tef1 gene promoter, the gene encoding the enzyme, alp gene terminator and pyrG marker gene linked to an in-fusion cloning site present in the multicloning site of pUC19.

In the case of inserting the gene by homologous recombination, one aspect of the DNA construct is a DNA construct containing a 5′ homologous recombination sequence, tef1 gene promoter, the gene encoding the enzyme, alp gene terminator and pyrG marker gene, and a 3′ homologous recombination sequence linked to each other.

In the case of inserting the gene by homologous recombination and recycling a marker, one form of the DNA construct is a DNA construct containing a 5′ homologous recombination sequence, tef1 gene promoter, the gene encoding the enzyme, alp gene terminator, a homologous sequence for marker recycling, pyrG marker gene, and a 3′ homologous recombination sequence linked to each other.

When the host organism is a filamentous fungus, a method known to those skilled in the art can be appropriately selected as a method for transforming the filamentous fungus. For example, a protoplast PEG method of preparing a protoplast of the host organism and then using polyethylene glycol and calcium chloride (see e.g., Mol. Gen. Genet. 218, 99-104, 1989 (supra); and Japanese Patent Laid-Open No. 2007-222055) can be used. An appropriate medium for regenerating the transformant is used according to the host organism used and the transformation marker gene. In the case of using, for example, A. oryzae or A. sojae as the host organism and pyrG gene as the transformation marker gene, the transformant can be regenerated in, for example, Czapek-Dox minimum medium (manufactured by Difco Laboratories Ltd.) containing 0.5% agar and 1.2 M sorbitol.

In order to obtain the transformant, for example, the promoter of the gene encoding the enzyme, originally carried by a chromosome of the host organism may be substituted by a high-expression promoter such as tef1 through the use of homologous recombination. In this respect, it is also preferred to insert a transformation marker gene such as pyrG, in addition to the high-expression promoter. For example, a cassette for transformation consisting of upstream region of the gene encoding the enzyme-transformation marker gene-high-expression promoter-whole or partial gene encoding the enzyme can be used for this purpose with reference to Examples described in Japanese Patent Laid-Open No. 2011-239681. In this case, the upstream region of the gene encoding the enzyme and the whole or partial gene encoding the enzyme are used for homologous recombination.

The whole or partial gene encoding the enzyme used can contain a sequence from a start codon to a midstream region. The length of the region suitable for homologous recombination is preferably 0.5 kb or larger.

The prepared transformant can be confirmed by culturing the transformant under conditions under which the enzymatic activity of the enzyme is observed, and subsequently detecting the product of interest in the cultures thus obtained by culture.

Alternatively, the prepared transformant may be confirmed by extracting chromosomal DNA from the transformant, and performing PCR with this chromosomal DNA as a template to confirm that a PCR product amplifiable by transformation is formed. In this case, the formation of a product having an expected length is confirmed by PCR using, for example, a forward primer against the nucleotide sequence of the promoter used and a reverse primer against the nucleotide sequence of the transformation marker gene in combination.

In the case of performing transformation by homologous recombination, it is preferred that the formation of a product having a length expected from homologous recombination be confirmed by PCR using a forward primer located upstream of the upstream homologous region used and a reverse primer located downstream of the downstream homologous region used in combination.

(Host Organism)

The host organism is not particularly limited as long as the organism can produce the enzyme by transformation with a DNA construct containing the gene encoding the enzyme. Examples thereof include microbes and plants. Examples of the microbe include microbes of the genus Aspergillus, microbes of the genus Escherichia, microbes of the genus Saccharomyces, microbes of the genus Pichia, microbes of the genus Schizosaccharomyces, microbes of the genus Zygosaccharomyces, microbes of the genus Trichoderma, microbes of the genus Penicillium, microbes of the genus Rhizopus, microbes of the genus Neurospora, microbes of the genus Mucor, microbes of the genus Acremonium, microbes of the genus Fusarium, microbes of the genus Neosartorya, microbes of the genus Byssochlamys, microbes of the genus Talaromyces, microbes of the genus Ajellomyces, microbes of the genus Paracoccidioides, microbes of the genus Uncinocarpus, microbes of the genus Coccidioides, microbes of the genus Arthroderma, microbes of the genus Trichophyton, microbes of the genus Exophiala, microbes of the genus Capronia, microbes of the genus Cladophialophora, microbes of the genus Macrophomina, microbes of the genus Leptosphaeria, microbes of the genus Bipolaris, microbes of the genus Dothistroma, microbes of the genus Pyrenophora, microbes of the genus Neofusicoccum, microbes of the genus Setosphaeria, microbes of the genus Baudoinia, microbes of the genus Gaeumannomyces, microbes of the genus Marssonina, microbes of the genus Sphaerulina, microbes of the genus Sclerotinia, microbes of the genus Magnaporthe, microbes of the genus Verticillium, microbes of the genus Pseudocercospora, microbes of the genus Colletotrichum, microbes of the genus Ophiostoma, microbes of the genus Metarhizium, microbes of the genus Sporothrix, microbes of the genus Sordaria, and plants of the genus Arabidopsis, and microbes and plants are preferred. However, human is excluded from the host organism in every case.

Among the filamentous fungi, for example, microbes of the genus Aspergillus such as A. oryzae, A. sojae, A. niger, A. tamarii, A. awamori, A. usamii, A. kawachii, and A. saitoi are preferred in light of safety and easy culture.

In the present embodiment, the expression of the protein is not limited to embodiments using the host organism as described above. For example, an in vitro cell-free protein expression system can be suitably used, particularly, when large-scale production such as production in a commercial scale is not intended. The cell-free protein expression system does not require cell culture and also has the advantage that the protein can also be conveniently purified. In the cell-free protein expression system, a reaction liquid containing a gene corresponding to the desired protein and molecular mechanisms of transcription and translation such as a cell lysate is used.

(Specific Example of Gene Encoding Enzyme)

Examples of the gene encoding the enzyme derived from the genus Sarocladium include genes g4462 and g10122 having the nucleotide sequences as set forth in SEQ ID NOs: 1 and 3, respectively. The amino acid sequences of the pentosidine oxidase 1 protein (PenOX1) and the pentosidine oxidase 2 protein (PenOX2) are shown in SEQ ID NO: 2 and SEQ ID NO: 4, respectively.

The method for obtaining the gene encoding the enzyme from the genus Sarocladium or an organism other than the genus Sarocladium is not particularly limited. The gene can be obtained, for example, by searching the genomic DNA of the target organism by BLAST homology search on the basis of the nucleotide sequences of the genes g4462 and g10122 (SEQ ID NO: 1 and SEQ ID NO: 3), and identifying genes having nucleotide sequences having high sequence identity to the nucleotide sequences of the genes g4462 and g10122. Also, the gene can be obtained by identifying proteins having amino acid sequences having high sequence identity to the amino acid sequences of the pentosidine oxidase 1 and pentosidine oxidase 2 proteins (SEQ ID NO: 2 and SEQ ID NO: 4) on the basis of the total protein of the target organism, and identifying genes encoding the proteins.

The gene encoding the enzyme obtained from the genus Sarocladium or the gene encoding an enzyme having sequence identity to the enzyme can be transferred to arbitrary host cells of a host organism such as a microbe of the genus Aspergillus for transformation.

(Transformant)

One form of the transformant is a transformant having an insert of any one of the genes or a combination thereof in a host organism such as a microbe or a plant transformed so as to express the inserted gene.

Another form of the transformant is a transformant having an insert of a DNA construct designed to highly or low express a gene (also containing a promoter sequence, etc. in addition to the ORF) containing the whole or a portion of the gene g4462 or g10122, and a transcriptional factor that controls the transcription of the gene, in a host organism such as a microbe or a plant transformed so as to express the inserted gene.

When the host organism is an organism found to have the ability to produce pentosidine oxidase, such as the genus Sarocladium, it is desirable that the inserted gene should be forced to be constitutively expressed or more highly expressed than endogenous expression, or should be conditionally expressed at the late stage of culture after cell proliferation. Such a transformant is cultured or grown under conditions suitable for the host organism or the transformant and can thereby produce pentosidine oxidase that is not produced in the host organism or a more detectable level of pentosidine oxidase than that produced in the host organism, through the action of the transcriptional factor having a changed expression level.

The pentosidine oxidase can be produced by culturing the transformant described above under culture conditions suitable for the growth of the transformant using a medium suitable for the growth of the transformant. The culture method is not particularly limited. When the host organism is, for example, a filamentous fungus, examples thereof include a solid culture method and a liquid culture method which are performed under draft or non-draft conditions. Hereinafter, a production method using a filamentous fungus as a host organism or a wild-type organism will be mainly described. However, the present embodiment is not limited by the following description.

Any synthetic medium or natural medium can be used as long as the medium is a usual medium for the culture of a host organism or a wild-type organism (hereinafter, these organisms are also collectively referred to as a “host organism, etc.”), i.e., contains a carbon source, a nitrogen source, an inorganic material, and other nutrients at an appropriate ratio. When the host organism, etc. is a microbe of the genus Aspergillus, YMG medium, PPY medium, or the like as described in Examples mentioned later can be used, though the medium is not particularly limited thereto.

Usual culture conditions for the host organism, etc. known to those skilled in the art can be adopted as culture conditions for the transformant. When the host organism, etc. is, for example, a filamentous fungus, the initial pH of the medium is adjusted to 5 to 10, and the culture temperature and the culture time can be appropriately set to 20 to 40° C. and, for example, several hours to several days, preferably 1 to 7 days, more preferably 2 to 4 days, respectively. The culture approach is not particularly limited, and aeration-stirring submerged culture, shake culture, static culture, or the like can be adopted. The culture is preferably performed under conditions that attain sufficient dissolved air. For example, in the case of culturing a microbe of the genus Aspergillus, one example of the medium and the culture conditions includes shake culture at 160 rpm at 30° C. for 3 to 5 days using YMG medium or PPY medium described in Examples mentioned later.

The method for extracting pentosidine oxidase from the cultures after the completion of culture is not particularly limited. For the extraction, a fungus body recovered by operation such as filtration or centrifugation from the cultures may be used as it is, or a fungus body dried after recovery or a further pulverized fungus body may be used. Examples of the method for drying the fungus body include, but are not particularly limited to, freeze drying, solar drying, hot-air drying, vacuum drying, through-flow drying, and drying under reduced pressure.

Instead of the treatments described above, fungus body disruption treatment may be adopted, for example, a method of destroying the fungus body using a destruction approach such as a sonicator, a French press, Dyno-Mill, or a mortar; a method of lysing the cell wall of the fungus body using a cell wall lytic enzyme such as yatalase; or a method of lysing the fungus body using a surfactant such as SDS or Triton X-100. These methods can be used singly or in combinations.

The obtained extracts can be subjected to a purification procedure such as centrifugation, filtration through a filter, ultrafiltration, gel filtration, separation based on difference in solubility, solvent extraction, chromatography (adsorption chromatography, hydrophobic chromatography, cation-exchange chromatography, anion-exchange chromatography, reverse-phase chromatography, etc.), crystallization, activated carbon treatment, or membrane treatment to purify the product of interest.

(Substrate Specificity)

The protein having activity that oxidatively degrades pentosidine according to the present embodiment has high degrading activity (substrate specificity) against pentosidine and may have degrading activity against other amino acids. In one aspect, the relative activity of the protein having activity that oxidatively degrades pentosidine according to the present embodiment against one or more, two or more, three or more, four or more or five or more amino acids selected from arginine, leucine, methionine, phenylalanine, tryptophan and tyrosine, or all of these amino acids is 40% or higher, 50% or higher or 60% or higher when the activity against pentosidine is defined as 100%. In one aspect, the activity of the protein having activity that oxidatively degrades pentosidine according to the present embodiment against one or more or two or more amino acids selected from asparagine, glutamine and histidine, or all of these amino acids has 10% or higher or 20% or higher relative activity when the activity against pentosidine is defined as 100%.

The methods for measuring the relative activity and the substrate specificity can be carried out by use of an approach known to those skilled in the art using the same or similar approaches and conditions as in measurement for pentosidine. They can be measured, for example, using a reaction rate with reference to an approach described in Examples mentioned later.

(Measurement Method)

The method for measuring pentosidine according to the present embodiment comprises the steps of:

degrading a specimen with an amino acid degrading enzyme;

contacting the specimen after the degradation step with a protein having activity that oxidatively degrades pentosidine; and

detecting change resulting from the contact.

As used herein, examples of the “specimen” include test subjects, for example, pentosidine solutions containing pentosidine to be quantified; and liquid components and solid components derived from organisms, such as blood, blood components (serum, plasma, blood cells, etc.), body fluids, and excrements. In one aspect, the specimen is derived from a subject having or suspected of having a disease associated with pentosidine. The specimen is preferably blood or a blood component, particularly preferably plasma. The specimen may not always contain pentosidine. If the specimen contains no pentosidine, the measurement method according to the present embodiment can be used in analysis on the presence or absence of contained pentosidine (qualitative analysis). When the specimen is derived from an organism, a specimen derived from an arbitrary organism such as a human, a mouse, a rat, or a monkey can be used. The specimen collected from the organism may be used as it is or may be used after arbitrary treatment.

(Amino Acid Degrading Enzyme)

The measurement method of the present embodiment comprises the step of degrading the specimen with an amino acid degrading enzyme. The degradation with the amino acid degrading enzyme prior to contact with a protein having activity that oxidatively degrades pentosidine decreases measurement errors ascribable to the reaction of the protein having activity that oxidatively degrades pentosidine with another amino acid and allows more accurate measurement of pentosidine.

The amino acid degrading enzyme is an enzyme different from the protein having activity that oxidatively degrades pentosidine and is an enzyme that can preferentially degrade an amino acid other than an amino acid of pentosidine. Examples of the amino acid degrading enzyme include amino acid oxidase, amino acid dehydrogenase, amino acid aminotransferase, amino acid decarboxylase, amino acid ammonia lyase, amino acid oxygenase (hydroxylase), and amino acid hydrolase. An arbitrary enzyme can be used taking its substrate specificity into consideration. These amino acid degrading enzymes may be used singly or as a mixture or in combination of two or more thereof.

The amino acid degrading enzyme is not particularly limited, and a known enzyme can be used. A commercially available product may be used. For example, a reagent of a commercially available amino acid quantification kit may be used as the amino acid degrading enzyme. The production method therefor is not limited. For example, a transformant harboring a gene encoding the amino acid degrading enzyme (as one example, a gene encoding escapin (SEQ ID NO: 15: the amino acid sequence of a mature peptide of escapin derived from Aplysia californica) described in Example 11 mentioned later) is prepared by use of the same or similar approach as mentioned above about the production of pentosidine oxidase, and this transformation is cultured. The amino acid degrading enzyme may be obtained from the resulting medium.

For example, amino acid degrading enzymes shown in the following table can be used singly or in combinations taking substrate specificity into consideration.

TABLE 1

| Large | Middle | | | |
|---|---|---|---|---|
| classification | classification | Small classification | Manufacturer | Model |
| Amino acid | L-Amino acid | Crotalus atorx-derived L-amino acid oxidase, | Merck | A5147 |
| oxidase | oxidase | Type I | | |
| | | Crotalus adamanteus-derived L-amino acid | Merck | A9253 |
| | | oxidase, Type I | | |
| | | Crotalus adamanteus-derived L-amino acid | Merck | A9378 |
| | | oxidase, Type IV | | |
| | | Aplysia californica-derived amino acid oxidase, | — | — |
| | | (Escapin) | | |
| | | Trichoderma viride-derived lysine oxidase | Merck | L6150 |
| Amino acid | Glutamate | Microbe-derived glutamate dehydrogenase | Toyobo | GTD-211 |
| dehydrogenase | dehydrogenase | | Co., Ltd. | |
| | | Bacillus subtilis-derived glutamate | — | — |
| | | dehydrogenase | | |
| | Leucine | Bacillus stearothermophilus-derived leucine | NIPRO | — |
| | dehydrogenase | dehydrogenase (LeuDH) | ENZYMES | |
| | | Bacillus sp.-derived leucine dehydrogenase | Toyobo | LED-201 |
| | | | Co., Ltd. | |
| | Alanine | Bacillus stearothermophilus-derived alanine | NIPRO | — |
| | dehydrogenase | dehydrogenase (AlaDH) | ENZYMES | |
| | | Streptomyces griseus-derived alanine | — | — |
| | | dehydrogenase | | |
| Amino acid | Alanine | Human liver-derived alanine aminotransferase | LEE | 310-19 |
| aminotransferase | aminotransferase | (ALT/GPT) | | |
| | | Oryza sativa-derived alanine aminotransferase | — | — |
| | Aspartate | Human liver-derived aspartate aminotransferase | LEE | 306-10 |
| | aminotransferase | (AST/GOT) | | |
| | | Swine heart-derived aspartate aminotransferase | LEE | 300-20 |
| | | (AST/GOT) | | |
| Amino acid | Glutamate | Lactobacillus brevis IFO 12005-derived | — | — |
| decarboxylase | decarboxylase | glutamate decarboxylase | | |
| | | Escherichia coli-derived glutamate | — | — |
| | | decarboxylase | | |
| | | Solanum lycopersicum-derived glutamate | — | — |
| | | decarboxylase | | |
| | Aspartate | Tetragenococcus halophilus-derived aspartate | — | — |
| | decarboxylase | decarboxylase | | |
| | | Corynebacterium glutamicum-derived aspartate | — | — |
| | | decarboxylase | | |
| | Arginine | Lactococcus lactis-derived arginine | — | — |
| | decarboxylase | decarboxylase | | |
| | | Lactobacillus sake-derived arginine | — | — |
| | | decarboxylase | | |
| | | Escherichia coli-derived arginine decarboxylase | — | — |
| | Lysine | Escherichia coli-derived lysine decarboxylase | — | — |
| | decarboxylase | Streptomyces coelicolor-derived lysine | — | — |
| | | decarboxylase | | |
| | Histidine | Lactobacillus buchneri-derived histidine | — | — |
| | decarboxylase | decarboxylase | | |
| | | Lactobacillus 30a-derived histidine | — | — |
| | | decarboxylase | | |
| | Phenylalanine | Lactobacillus buchneri-derived phenylalanine | — | — |
| | decarboxylase | decarboxylase | | |
| | | Pseudomonas putida-derived phenylalanine | — | — |
| | | decarboxylase | | |
| | Tyrosine | Lactobacillus buchneri-derived tyrosine | — | — |
| | decarboxylase | decarboxylase | | |
| | | Nicotiana tabacum-derived tyrosine | — | — |
| | | decarboxylase | | |
| | Tryptophan | Oryza sativa-derived tryptophan decarboxylase | — | — |
| | decarboxylase | Nicotiana tabacum-derived tryptophan | — | — |
| | | decarboxylase | | |
| Amino acid | Histidine | Pseudomonas putida-derived histidine ammonia | — | — |
| ammonia lyase | ammonia lyase | lyase | | |
| | | Streptomyces griseus-derived histidine ammonia | — | — |
| | | lyase | | |
| | Phenylalanine | Rhodotorula rubra-derived phenylalanine | — | — |
| | ammonia lyase | ammonia lyase | | |
| | | Rhodobacter capsulatus-derived phenylalanine | — | — |
| | | ammonia lyase | | |
| | Tyrosine | Rhodotorula glutinis-derived tyrosine ammonia | — | — |
| | ammonia lyase | lyase | | |
| | | Saccharothrix espanaensis-derived tyrosine | — | — |
| | | ammonia lyase | | |
| Amino acid | Tryptophan | Bacillus megaterium-derived tryptophan | — | — |
| oxygenase | dioxygenase | dioxygenase | | |
| (hydroxylase) | Asparagine | Streptomyces coelicolor A3(2)-derived | — | — |
| | hydroxylase | asparagine hydroxylase | | |
| Aminoa cid | Arginase | Saccharomyces cerevisiae-derived arginase | — | — |
| hydrolase | | Solanum lycopersicum-derived arginase | — | — |
| | Glutaminase | Aspergillus oryzae-derived glutaminase | — | — |
| | | Micrococcus luteus-derived glutaminase | — | — |
| | Asparaginase | Aspergillus oryzae-derived asparaginase | — | — |
| | | Escherichia coli-derived asparaginase | — | — |

The amino acid degrading enzyme is an enzyme capable of degrading the desired amino acid under conditions unreactive with pentosidine. In one aspect, the relative activity of the amino acid degrading enzyme against pentosidine is 30% or lower, preferably 20% or lower, more preferably 10% or lower, further preferably 5% or lower, when the activity against an amino acid against which the highest activity is exerted (which is most degraded) is defined as 100% under the same conditions thereas.

The amino acid degrading enzyme can be selected taking into consideration the type of an amino acid presumably contained in the specimen to be measured, or the substrate specificity of the protein having activity that oxidatively degrades pentosidine for use in measurement.

In one aspect, an amino acid degrading enzyme that degrades an amino acid against which the protein having activity that oxidatively degrades pentosidine has 5% or higher, 10% or higher, 20% or higher, 40% or higher or 60% or higher relative activity can be used, when the activity of the protein having activity that oxidatively degrades pentosidine against pentosidine is defined as 100%.

In one aspect, when the protein having activity that oxidatively degrades pentosidine is pentosidine oxidase (preferably pentosidine oxidase having the amino acid sequence of SEQ ID NO: 2 or SEQ ID NO: 4 or a variant thereof, more preferably pentosidine oxidase having the amino acid sequence of SEQ ID NO: 4 or a variant thereof), an amino acid degrading enzyme that degrades one or more, two or more, three or more, four or more or five or more amino acids selected from arginine, leucine, methionine, phenylalanine, tryptophan and tyrosine, or preferably all of these amino acids, can be used. For example, any of the following amino acid degrading enzymes can be used as such an amino acid degrading enzyme.

An amino acid degrading enzyme having 30% or lower, preferably 20% or lower, more preferably 10% or lower, further preferably 5% or lower relative activity against pentosidine when the activity against one or more amino acids selected from arginine, leucine, methionine, phenylalanine, tryptophan and tyrosine is defined as 100% under the same conditions thereas.

A combination of amino acid degrading enzymes, the combination having 30% or lower, preferably 20% or lower, more preferably 10% or lower, further preferably 5% or lower relative activity against pentosidine when the activity of the combined enzymes against arbitrary amino acid(s) selected from arginine, leucine, methionine, phenylalanine, tryptophan and tyrosine is defined as 100% under the same conditions thereas.

A combination of escapin or a variant thereof that exhibits substrate specificity similar thereto, and Crotalus adamanteus-derived L-amino acid oxidase or a variant thereof that exhibits substrate specificity similar thereto.

A combination of escapin or a variant thereof that exhibits substrate specificity similar thereto, Crotalus adamanteus-derived L-amino acid oxidase or a variant thereof that exhibits substrate specificity similar thereto, and further, one or more, two or more, three or more, four or more or all enzymes selected from histidine decarboxylase, asparaginase, aspartic acid decarboxylase, glutaminase and glutamic acid decarboxylase.

In one aspect, when the protein having activity that oxidatively degrades pentosidine is pentosidine oxidase (preferably pentosidine oxidase having the amino acid sequence of SEQ ID NO: 2 or SEQ ID NO: 4 or a variant thereof, more preferably pentosidine oxidase having the amino acid sequence of SEQ ID NO: 4 or a variant thereof), an amino acid degrading enzyme that degrades one or more or two or more amino acids selected from asparagine, glutamine and histidine, or all of these amino acids, in addition to arginine, leucine, methionine, phenylalanine, tryptophan and tyrosine, can be used.

The conditions under which the specimen is degraded with the amino acid degrading enzyme are not particularly limited as long as under the conditions, the amino acid degrading enzyme can degrade the desired amino acid and does not react with pentosidine. The conditions can be appropriately set according to the amino acid degrading enzyme used.

In the case of using, for example, amino acid oxidase as the amino acid degrading enzyme, its amount can be appropriately selected depending on the amount of the amino acid contained in the specimen, reaction conditions, etc., and is 0.001 to 50 U/ml, preferably 0.01 to 10 U/ml. The pH can be adjusted to, for example, pH 3 to 12, preferably pH 4 to 11, taking into consideration the range that allows the amino acid oxidase used to act. An arbitrary known pH adjuster and buffer solution can be used according to the specimen and the pH to be adjusted. For example, 15 to 65° C., preferably 20 to 60° C., can be adopted as a reaction temperature taking into consideration the optimum temperature range of the amino acid oxidase used. The reaction time can be a time sufficient for degrading the desired amino acid, and the reaction can be performed for, for example, 1 to 120 minutes, preferably 2 to 60 minutes.

After the degradation step, the specimen thus degraded with the amino acid degrading enzyme is contacted, either as it is or after appropriately undergoing, if necessary, a step such as heating, centrifugation, concentration, or dilution, with the protein having activity that oxidatively degrades pentosidine.

The conditions under which the specimen is contacted with the protein having activity that oxidatively degrades pentosidine are not particularly limited as long as under the conditions, pentosidine can be degraded.

In the case of using, for example, pentosidine oxidase as the protein having activity that oxidatively degrades pentosidine, its amount is appropriately selected depending on the amount of pentosidine that may be contained in the specimen, reaction conditions, etc., and is 0.001 to 50 U/ml, preferably 0.01 to 10 U/ml. The pH can be adjusted to, for example, pH 4 to 10, preferably pH 5.5 to 9, taking into consideration the range that allows the pentosidine oxidase used to act. An arbitrary known pH adjuster and buffer solution can be used according to the specimen and the pH to be adjusted. For example, 20 to 60° C., preferably 30 to 55° C., can be adopted as a reaction temperature taking into consideration the optimum temperature range of the pentosidine oxidase used. The reaction time can be a time sufficient for degrading the desired amino acid, and the reaction can be performed for, for example, 1 to 120 minutes, preferably 2 to 60 minutes.

Then, change resulting from the contact is detected. As used herein, the “change resulting from the contact” means the presence or absence of a starting material such as pentosidine contained in the specimen, a reaction product with the protein having activity that oxidatively degrades pentosidine or a material consumed through the reaction, etc., or time-dependent change in amount thereof.

In a more specific aspect, the method for measuring pentosidine may comprise the steps of:

(A) allowing pentosidine oxidase to act on a specimen in the presence of water and oxygen; and

(B) measuring an amount of at least one of a reaction product and a material consumed through the reaction through the action of the pentosidine oxidase.

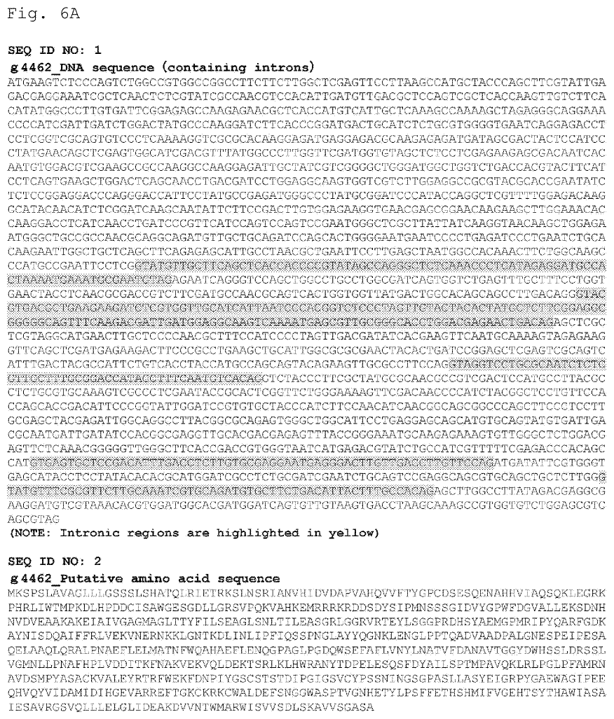

Examples of the reaction product to be measured in the step (B) can include hydrogen peroxide, ammonia and pentosidine deamination products. The amount of the reaction product hydrogen peroxide can be measured, for example, through peroxidase reaction. The amount of the reaction product ammonia can be measured by, for example, a method using an indophenol method or Nessler's reagent, or a method of measuring the amount of NADH using an enzyme for ammonia as a substrate, such as glutamic acid dehydrogenase or NAD synthase. As used herein, the “deamination product” means, for example, a product having keto acid at at least one of the ends by the removal of one or both of the amino groups of lysine and arginine constituting pentosidine and the replacement thereof with oxygen. FIG. 5 shows an example of such a deamination product. Examples of the material consumed through the reaction to be measured in the step (B) can include oxygen. The amount of oxygen decreased through enzyme reaction can be measured using, for example, an oxygen electrode. Alternatively, the amount may be colorimetrically determined by oxidizing a manganese ion with oxygen on the basis of the Winkler approach.

It has been reported that plasma has a pentosidine concentration higher by approximately 70% in, for example, schizophrenia patients (68.4 ng/mL) compared with healthy individuals (mean±S.D.: 39.6±7.8 ng/mL) (Arai et al., Psychiatria et Neurologia Japonica (2012), Vol. 114, No. 2, pp. 101-107; and Arai, M., et al. Arch Gen Psychiat, 67; 589-597, 2010). According to the measurement method of the present embodiment, the degradation with the amino acid degrading enzyme prior to contact with the protein having activity that oxidatively degrades pentosidine decreases measurement errors ascribable to the reaction of the protein having activity that oxidatively degrades pentosidine with another amino acid to less than 70%, preferably less than 60%, more preferably less than 50%, and allows more accurate measurement of pentosidine. Therefore, the measurement method of the present embodiment is also very useful in the diagnosis of a disease associated with pentosidine.

In another aspect, the present embodiment provides a kit for measuring pentosidine in a specimen, comprising: an amino acid degrading enzyme and a protein having activity that oxidatively degrades pentosidine. The kit according to the present embodiment can be used for detecting a reaction product of pentosidine and the protein having activity that oxidatively degrades pentosidine, or a material consumed through the reaction. The kit according to the present embodiment may further contain at least one of a buffer solution for reaction, a reagent for reaction product detection, for example, a reagent for hydrogen peroxide detection, a reagent for ammonia detection and a reagent for pentosidine deamination product detection, and a reagent for detection of a material consumed through the reaction, for example, a reagent for oxygen detection. The kit of the present embodiment may be used as an ex vivo diagnostic drug and can be suitably used, for example, in the diagnosis of a disease associated with pentosidine or a reaction product of pentosidine and pentosidine oxidase, for example, diabetes mellitus or nephropathy.

Examples of the reagent for hydrogen peroxide detection include 10-(carboxymethylaminocarbonyl)-3,7-bis(dimethylamino)-phenothiazine (DA-67) and N-(carboxymethylaminocarbonyl)-4,4′-bis(dimethylamino)-diphenylamine (DA-64), which can detect hydrogen peroxide with high sensitivity, as well as known chromogenic reagents such as Trinder's reagent. Examples of the reagent for ammonia detection include a combination of phenol-sodium nitroprusside and an oxidizing agent such as sodium hypochlorite (indophenol method), and Nessler's reagent. Examples of the reagent for oxygen detection include manganese ions and a combination of sodium hydroxide and sulfuric acid.

The detection of the reaction product using chromogenic reaction can be performed very conveniently and inexpensively as compared with an immunochemical method or an instrumental analytical approach. However, the detection of the reaction product or the material consumed through the reaction does not exclude other known quantitative or qualitative methods except for detection reagents, and any of the methods may be appropriately adopted. The detection may be performed using, for example, an apparatus such as an enzyme sensor equipped with a dedicated detection electrode, instead of the hydrogen peroxide or ammonia detection reagent.

The method for detecting the reaction product or the material consumed through the reaction may also be used in a method for detecting a disease associated directly or indirectly with pentosidine or each reaction product or material consumed through the reaction, and by extension, a method for diagnosing the disease.

The present embodiment further relates to a method for producing a reaction product of pentosidine derived from a specimen, comprising the steps of:

degrading the specimen with an amino acid degrading enzyme; and

contacting the specimen after the degradation step with a protein having activity that oxidatively degrades pentosidine, wherein

the amino acid degrading enzyme and the protein having activity that oxidatively degrades pentosidine are different from each other.

Each step can be carried out with reference to the description about the method for measuring pentosidine.

Hereinafter, the present embodiment will be described in more detail with reference to Examples. However, the present invention is not limited by these Examples. The present invention can assume various forms as long as the object of the present invention is attained.

### EXAMPLES

### (Example 1) Methods for Culturing Sarocladium sp. and Preparing Enzyme Liquid

Medium Used

MEA medium: Malt extract agar (manufactured by Oxoid Ltd.) was dissolved in distilled water into 50 g/L.

YMG medium: 0.4% yeast extract, 1% malt extract, and 0.4% glucose, pH 5.5

Culture of Fungus Strain

Sarocladium sp. F10012 strain preserved at −80° C. was applied to MEA medium and statically cultured at 24° C. for 7 to 10 days until a sufficient amount of hypha was obtained. The obtained hypha was inoculated to 250 mL of YMG medium in a 1 L flask and shake-cultured at 30° C. for 3 days.

Preparation of Crude Enzyme Liquid

The YMG medium containing the cultured fungus body was filtered using Miracloth (manufactured by Merck Millipore) for the removal of the fungus body to obtain a culture supernatant. The process of concentrating the culture supernatant using an ultrafiltration membrane (Vivaspin 20-3k, manufactured by GE Healthcare Japan Corp.), and diluting the concentrate with a 50 mM potassium phosphate buffer (pH 7.5) was repeated a plurality of times to replace the YMG medium with the potassium phosphate buffer while removing small molecules.

Partial Purification of Enzyme of Interest

The buffer-replaced crude enzyme liquid was fractionated using a column for ion-exchange chromatography (HiTrap Q Sepharose Fast Flow 1 mL, manufactured by GE Healthcare Japan Corp.). Specific procedures are as follows.

First, the crude enzyme liquid was loaded onto a column equilibrated with a 50 mM potassium phosphate buffer (pH 7.5) so that the enzyme was adsorbed onto the column. Then, the column was washed with 5 mL of a potassium phosphate buffer to elute unadsorbed proteins.

Then, 5 mL each of buffers containing 0.25 M, 0.5 M, 0.75 M, or 1.0 M sodium chloride dissolved in a potassium phosphate buffer was sequentially passed through the column to elute proteins adsorbed on the column.

A liquid eluted from the column upon loading of the crude enzyme liquid was designated as “Flow through”; a liquid eluted at the time of washing with the buffer was designated as “Start buffer”; and liquids eluted with the buffers containing sodium chloride were designated as “Elution 1”, “Elution 2”, “Elution 3” and “Elution 4”, respectively. These liquids were separately recovered into different containers.

### (Example 2) Method for Measuring Pentosidine Oxidase Activity

Activity Measurement of Partially Purified Enzyme Liquid

Each liquid eluted from the column for ion-exchange chromatography was used as a sample to measure activity. 50 μL of the sample was mixed with 25 μL of 4 mM pentosidine (in terms of a free form) (manufactured by Peptide Institute, Inc.; 3-trifluoroacetate (TFA) salt was used) dissolved in a 100 mM potassium phosphate buffer (pH 8.0) and 25 μL of an oxidase coloring reagent (4 U/mL peroxidase (manufactured by Toyobo Co., Ltd.), 1.8 mM 4-aminoantipyrine (manufactured by Fluka/Honeywell International Inc.), and 2 mM TOOS (manufactured by Dojindo Laboratories Co., Ltd.)), and the mixture was reacted at room temperature.

For the reaction, a 96-well microwell plate (manufactured by Nunc/Thermo Fisher Scientific, Inc.) was used. The blank used was supplemented with a 100 mM potassium phosphate buffer (pH 8.0) instead of a substrate solution. The absorbance at 555 nm of the reaction liquids and the blank solution was measured, and the strength of enzymatic activity was evaluated on the basis of difference in absorbance (40D).

Substrate Concentration Dependence Test

The pentosidine oxidase activity of each partially purified enzyme liquid was measured using varying concentrations of a substrate to evaluate change in activity against the concentrations of the substrate. The concentrations of the substrate solutions used were 0.13 mM, 0.25 mM, 0.5 mM, 1.0 mM, 2.0 mM and 4.0 mM.

Thermal Deactivation Test

Each partially purified enzyme liquid was heat-treated at 80° C. for 1 hour for protein denaturation. The pentosidine oxidase activity of this heat-treated sample was measured in accordance with the method for measuring activity mentioned above, and compared with the activity of an unheated sample.

### (Example 3) Pentosidine Oxidase Activity Analysis of Sarocladium sp. Enzyme Liquid

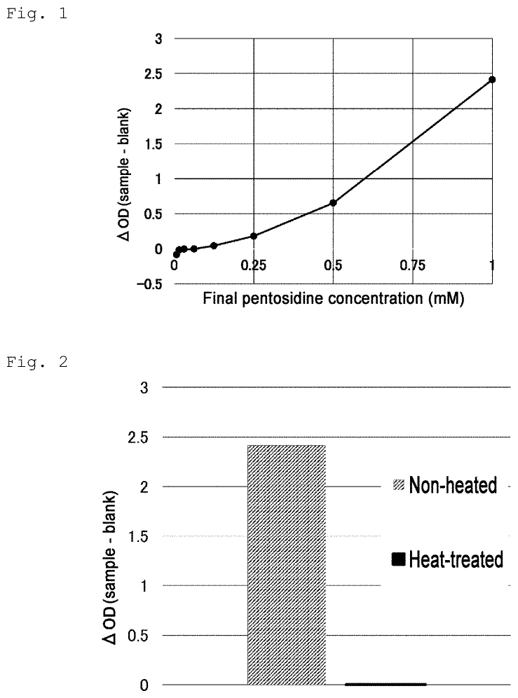

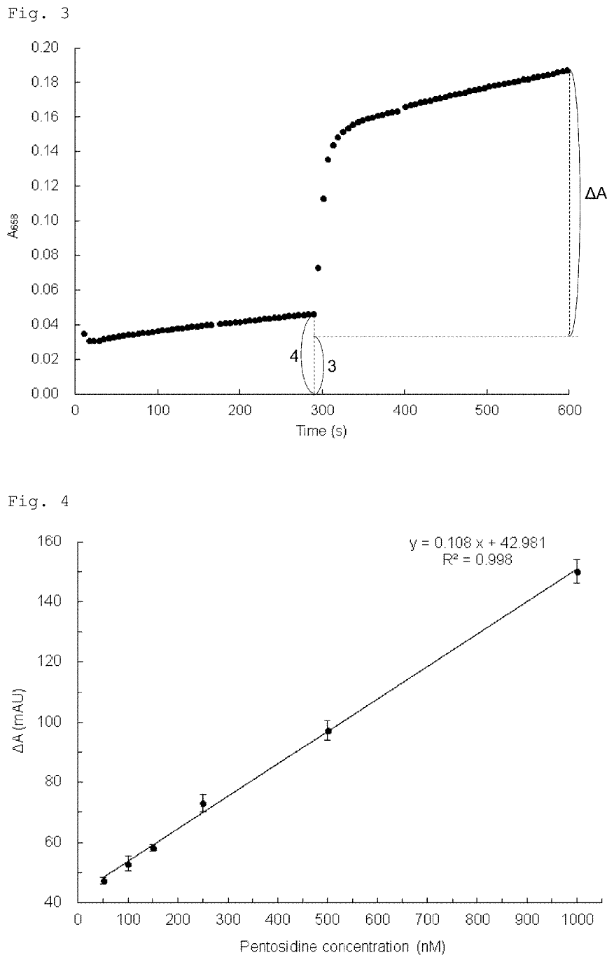

Each sample of the culture supernatant of Sarocladium sp. fractionated with a column for ion-exchange chromatography was analyzed for its reactivity with pentosidine. As a result, strong activity was observed in Elution 1 obtained by elution with the potassium phosphate buffer containing 0.25 M sodium chloride, suggesting that pentosidine oxidase was contained therein. As a result of subjecting this Elution 1 to the substrate concentration dependence test (FIG. 1) and the thermal deactivation test (FIG. 2), the enzymatic activity was found to be elevated in a substrate concentration-dependent manner and completely deactivated by heat treatment. This indicated that the pentosidine oxidase activity observed in Elution 1 was derived from the enzyme.

### (Example 4) Sequencing Pentosidine Oxidase Derived from Sarocladium sp.

Two types of putative pentosidine oxidase genes (SEQ ID NO: 1 and SEQ ID NO: 3) and amino acid sequences thereof (SEQ ID NO: 2 and SEQ ID NO: 4) were identified on the basis of the results described above and sequence information on the whole genome of Sarocladium sp.

### (Example 5) Heterologous Recombinant Expression of Pentosidine Oxidase Derived from Sarocladium sp. in Aspergillus sojae

In order to analyze the enzymatic activity of two pentosidine oxidases identified as described above, heterologous recombinant expression was performed with Aspergillus sojae as a host.

Preparation of Expression Vector

The codon-modified nucleotide sequences of SEQ ID NOs: 5 and 6 for expression in Aspergillus were each obtained by artificial gene synthesis on the basis of the amino acid sequences of SEQ ID NO: 2 and SEQ ID NO: 4.

For an expression cassette for expressing the pentosidine oxidase genes (penox1 and penox2) of SEQ ID NO: 5 and SEQ ID NO: 6, translation elongation factor gene tef1 promoter sequence Ptef (748-bp upstream region of the tef1 gene; SEQ ID NO: 7) was used as a promoter, and alkaline protease gene alp terminator sequence Talp (800-bp downstream region of the alp gene; SEQ ID NO: 8) was used as a terminator.

The selective marker used was transcription marker gene pyrG3 (1,487 bp including a 56-bp upstream region, an 896-bp coding region and a 535-bp downstream region; SEQ ID NO: 9) which complements uracil/uridine auxotrophy and allows multicopy transfer of a gene (see Japanese Patent Laid-Open No. 2018-068292). These Ptef, Talp, and pyrG3 sequences were obtained through PCR reaction with the genomic DNA of Aspergillus sojae NBRC4239 strain as a template.

Next, In-Fusion HD Cloning Kit (manufactured by Clontech Laboratories, Inc.) was used for linking these DNAs. For example, in the case of linking Ptef, the penox1 gene and Talp, DNA fragments were amplified through PCR reaction using the reverse primer of SEQ ID NO: 10 for Ptef and the forward primer of SEQ ID NO: 11 for Talp. In this respect, a 15-bp sequence complementary to the 5′ end of the penox1 gene (SEQ ID NO: 5) was added to the 5′ end of the reverse primer for Ptef amplification of SEQ ID NO: 10. A 15-bp sequence homologous to the 3′ end of the penox1 gene (SEQ ID NO: 5) was added to the 5′ end of the forward primer for Talp amplification of SEQ ID NO: 11. Therefore, Ptef, the penox1 gene and Talp can be linked through in-fusion reaction. In this way, expression vectors p19-pG3-penox1 and p19-pG3-penox2 were prepared in which Ptef-penox1-Talp-pyrG3 or Ptef-penox2-Talp-pyrG3 consisting of Ptef, the penox1 gene or the penox2 gene, Talp and pyrG3 linked in this order was inserted in the multicloning site of a pUC19 plasmid.

Preparation and Culture of Expressing Aspergillus Strain

A pyrG gene disruptant (strain deficient in a 48-bp upstream region, an 896-bp coding region and a 240-bp downstream region of the pyrG gene) of Aspergillus sojae was transformed by the protoplast PEG method using the plasmid p19-pG3-penox1 or p19-pG3-penox2 for transformation obtained as described above to obtain nine As-penox1 strain and six As-penox2 strain as Aspergillus sojae transformants having multicopy inserts of the expression cassette of penox1 or penox2.

Each of the obtained Aspergillus sojae transformants (As-penox1 strain and As-penox2 strain) was inoculated to 15 mL of PPY liquid medium (2% (w/v) Pinedex, 1% (w/v) Polypeptone, 0.5% (w/v) yeast extracts, 0.5% (w/v) monopotassium dihydrogen phosphate, and 0.05% (w/v) magnesium sulfate heptahydrate) contained in a 50 mL Erlenmeyer flask, and shake-cultured at 30° C. for 4 to 5 days.

Preparation of Hypha Extract

The culture liquid of each of the As-penox1 strain and the As-penox2 strain was filtered using Miracloth (manufactured by Merck Millipore) for the removal of the culture supernatant to obtain a fungus body. The fungus body was resuspended in 15 mL of a 10 mM potassium phosphate buffer (pH 7.5) and then disrupted using Micro Smash MS-100R (manufactured by Tomy Seiko Co., Ltd.). The fungus body homogenates were centrifuged at 15,000 rpm for 15 minutes to recover a supernatant as a crude enzyme liquid.

Measurement of L-Arginine Oxidizing Activity of Hypha Extract

200 μL of each crude enzyme liquid was mixed with 380 μL of a solution of 7.1 U/mL peroxidase, 0.70 mM 4-aminoantipyrine, and 0.79 mM TOOS dissolved in a 150 mM potassium phosphate buffer (pH 7.0), and the mixture was incubated at 37° C. for 5 minutes. Then, 20 μL of a 60 mM L-arginine solution was added thereto, and the mixture was stirred and reacted at 37° C. for 5 minutes. Time-dependent change in A₅₅₅ during the reaction was measured in a spectrophotometer (U-3900, manufactured by Hitachi High-Tech Science Corp.). A control experiment was carried out by adding 20 μL of ion-exchange water instead of 20 μL of the 60 mM L-arginine solution. The amount of the enzyme that produced 1 μmol of hydrogen peroxide per minute at 37° C. was defined as 1 unit (U). The activity was calculated according to the following expression.

Activity (U/mL)={(ΔAs−ΔA0)×0.6×df}/(39.2×0.5×0.2)

ΔAs: Amount of change in A₅₅₅ per minute of the reaction liquid

ΔA0: Amount of change in A₅₅₅ per minute of the control experiment

39.2: Millimolar extinction coefficient (mM⁻¹·cm⁻¹) of a quinonimine dye produced through reaction

0.5: Molar number of a quinonimine dye produced with 1 mol of hydrogen peroxide

0.6: Total volume (mL) of the reaction liquid

df: Dilution coefficient

0.2: Volume (mL) of the enzyme liquid

The L-arginine oxidizing activity of the crude enzyme liquids of the As-penox1 strain and the As-penox2 strain was 0.009 U/mL (As-penox1-15 strain) and 5.1 U/mL (As-penox2-16 strain), respectively, at maximum.

### (Example 6) Purification of Recombinant Penox2 Extracted from Hypha

The crude enzyme liquid of the As-penox2-16 strain was buffer-replaced with a 10 mM potassium phosphate buffer (pH 7.5) and then fractionated using an anion-exchange chromatography column (HiScreen Capto Q, manufactured by GE Healthcare Japan Corp.). First, the crude enzyme liquid was loaded onto a column equilibrated with a 10 mM potassium phosphate buffer (pH 7.5) so that the enzyme was adsorbed onto the column. Then, the column was washed with a 10 mM potassium phosphate buffer (pH 7.5) to elute unadsorbed proteins. Then, proteins adsorbed on the column were eluted while the concentration of sodium chloride contained in a 10 mM potassium phosphate buffer (pH 7.5) was linearly elevated from 0 mM to 40 mM. Fractions that exhibited L-arginine oxidizing activity were analyzed by SDS-PAGE to recover a fraction free from foreign proteins as purified PenOX2. The recovered purified PenOX2 solution was concentrated using an ultrafiltration membrane (Amicon Ultra 15-30k, manufactured by Merck KGaA) until the L-arginine oxidizing activity reached 24 U/mL, and used in a pentosidine quantification test.

### (Example 7) Pentosidine Quantification Test

The following reagents were prepared, and pentosidine was measured using Bio Majesty JCA-BM1650 (manufactured by JEOL Ltd.).

(Sample: Pentosidine Solution)

0.2 μM, 0.4 μM, 0.6 μM, 1.0 μM, 2.0 μM or 4.0 μM pentosidine solution (prepared using the same pentosidine as in Example 2)

(First Reagent: Leuco Dye, Peroxidase Solution)

120 mM potassium phosphate buffer (pH 7.0)

0.2 mM DA-67 (10-(carboxymethylaminocarbonyl)-3,7-bis(dimethylamino)phenothiazine, sodium salt) (manufactured by FUJIFILM Wako Pure Chemical Corp.)

3.0 U/mL peroxidase

(Second Reagent: PenOX2 Solution)

120 mM potassium phosphate buffer (pH 7.0)

24 U/mL PenOX2

25 μL of each sample was added to 50 μL of the first reagent, and the mixture was incubated at 37° C. for 5 minutes. Then, 25 μL of the second reagent was added thereto, and pentosidine oxidation reaction mediated by PenOX2 and reaction for the detection of hydrogen peroxide produced through the reaction were allowed to proceed at 37° C. for 5 minutes.

In the reaction for the detection of hydrogen peroxide, DA-67 was oxidized, with concomitant consumption of peroxidase, into methylene blue which in turn developed color while absorbance (A₆₅₈) was elevated. FIG. 3 shows, as one example, the relationship between the time elapsed from the mixing of the sample (4.0 μM pentosidine solution) with the first reagent and the absorbance (A₆₅₈). Elevation in A₆₅₈ was able to be confirmed immediately after addition of the second reagent containing PenOX2.

Subsequently, the amount of elevation in A₆₅₈ (ΔA) caused by the oxidation of pentosidine was calculated according to the following expression.

ΔA=(Absorbance 5 minutes after addition of the second reagent)−(Absorbance immediately before addition of the second reagent×0.75)

(Since the concentration of the composition in the reaction liquid was changed 0.75-fold (75/100-fold) by the addition of the second reagent,

the value obtained by multiplying the absorbance immediately before addition of the second reagent by 0.75 was regarded as absorbance immediately after addition of the second reagent.)

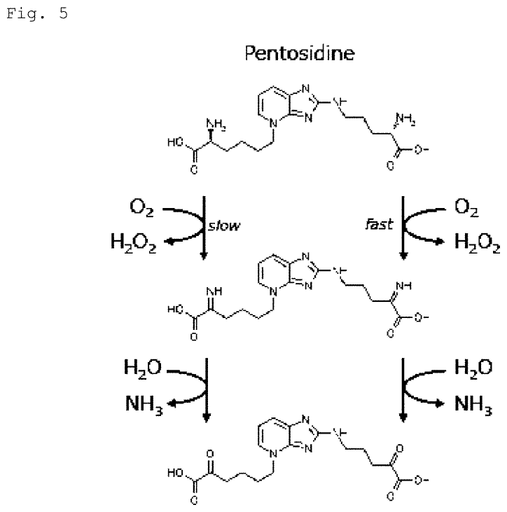

Correlation held true between the final pentosidine concentration and ΔA (FIG. 4). This indicated that PenOX2 exhibits pentosidine oxidizing activity and can be used in the quantification of pentosidine. Likewise, PenOX1 exhibited pentosidine oxidizing activity, though the results are not shown.

### (Example 8) Purification of Recombinant Penox2 Secreted from Hypha

The hypha culture liquid of the As-penox2 strain was filtered using Miracloth (manufactured by Merck Millipore) to recover a hypha culture supernatant. 75 mL of the obtained hypha culture supernatant was filtered through a syringe filter having a pore size of 0.2 μm and then concentrated using an ultrafiltration membrane (Amicon Ultra 15-30k, manufactured by Merck KGaA). Ammonium sulfate was gradually added to the concentrate so as to attain 70% saturation. The mixture was left at 4° C. for 2 hours and then centrifuged (15,000 rpm, 4° C., 5 min) to recover a supernatant while precipitating redundant proteins. The recovered supernatant was concentrated using an ultrafiltration membrane (Amicon Ultra 0.5-30 k, manufactured by Merck KGaA).

A 50 mM potassium phosphate buffer (pH 7.5) containing 2 M ammonium sulfate was added to this concentrate, and the mixture was then fractionated using a column for hydrophobic interaction chromatography (HiTrap Butyl Fast Flow 1 mL, manufactured by GE Healthcare Japan Corp.). Specific procedures are as follows.

First, the crude enzyme liquid was loaded onto a column equilibrated with a 50 mM potassium phosphate buffer (pH 7.5) containing 2 M ammonium sulfate so that the enzyme was adsorbed onto the column. Then, the column was washed with 10 mL of a 50 mM potassium phosphate buffer (pH 7.5) containing 2 M ammonium sulfate to elute unadsorbed proteins.

Then, 5 mL each of 50 mM potassium phosphate buffers (pH 7.5) containing 1.5 M, 1.3 M, or 1.15 M ammonium sulfate, 10 mL of a 50 mM potassium phosphate buffer (pH 7.5) containing 1 M ammonium sulfate, and 5 mL of a 50 mM potassium phosphate buffer (pH 7.5) free from ammonium sulfate were sequentially passed through the column to elute proteins adsorbed on the column.

A liquid eluted from the column upon loading of the crude enzyme liquid was designated as “Flow through 1”; a liquid eluted at the time of washing with the buffer containing 2 M ammonium sulfate was designated as “Elution 1”; liquids eluted with the buffers containing 1.5 M, 1.3 M, 1.15 M, or 1 M ammonium sulfate were designated as “Elution 2”, “Elution 3”, “Elution 4” and “Elution 5”, respectively; and a liquid eluted with the buffer free from ammonium sulfate was designated as “Elution 6”. These liquids were separately recovered into different containers.

Each fractionated sample was analyzed for its reactivity with pentosidine. As a result, strong activity was observed in Elution 5 obtained by elution with the potassium phosphate buffer containing 1 M ammonium sulfate, suggesting that pentosidine oxidase (PenOX2) was contained therein. This Elution 5 was concentrated using an ultrafiltration membrane (Amicon Ultra 15-30 k, manufactured by Merck KGaA), and the concentrate was buffer-replaced with a 50 mM potassium phosphate buffer (pH 7.5) free from ammonium sulfate and then concentrated again using an ultrafiltration membrane (Amicon Ultra 15-30 k, manufactured by Merck KGaA). This concentrate was fractionated using a column for ion-exchange chromatography (HiTrap Q Sepharose Fast Flow 1 mL, manufactured by GE Healthcare Japan Corp.). Specific procedures are as follows.

First, the crude enzyme liquid was loaded onto a column equilibrated with a 50 mM potassium phosphate buffer (pH 7.5) so that the enzyme was adsorbed onto the column. Then, the column was washed with 5 mL of a 50 mM potassium phosphate buffer (pH 7.5) to elute unadsorbed proteins.

Then, 1 mL (which was passed five times) of a liquid of 0.1 M sodium chloride dissolved in a 50 mM potassium phosphate buffer (pH 7.5), 1 mL (which was passed five times) of a liquid of 0.175 M sodium chloride dissolved in the buffer, and 5 mL (which was passed once) of a liquid of 1 M sodium chloride dissolved in the buffer were sequentially passed through the column to elute proteins adsorbed on the column.

A liquid eluted from the column upon loading of the crude enzyme liquid was designated as “Flow through 2”; a liquid eluted at the time of washing with the buffer was designated as “Elution 7”; and liquids eluted with the buffers containing sodium chloride were designated as “Elution 8-1”, “Elution 8-2”, “Elution 8-3”, “Elution 8-4”, “Elution 8-5”, “Elution 9-1”, “Elution 9-2”, “Elution 9-3”, “Elution 9-4”, “Elution 9-5” and “Elution 10”, respectively. These liquids were separately recovered into different containers.

Each fractionated sample was analyzed for its reactivity with pentosidine. As a result, strong activity was observed in Elution 9-1 and Elution 9-2 obtained by elution with the potassium phosphate buffer containing 0.175 M sodium chloride, suggesting that pentosidine oxidase was contained therein. As a result of analyzing a mixture of the active fractions Elution 9-1 and Elution 9-2 in equal amounts by SDS-PAGE, substantially a single band was obtained (molecular weight: approximately 80,000). The active fractions thus obtained were used for determining the following physicochemical properties.

### (Example 9) Physicochemical Property of PenOX2 Produced from Aspergillus sojae Transformant as-Penox2 Strain

In order to determine the physicochemical properties of PenOX2, the following method for measuring enzymatic activity was used.

600 μL of an arbitrary buffer, 400 μL of a solution of 3.99 U/mL peroxidase, 1.8 mM 4-aminoantipyrine, and 2 mM TOOS dissolved in deionized water, and 150 μL of deionized water were incubated at an arbitrary temperature for 10 minutes. Then, 50 μL of the enzyme liquid preserved on ice and 400 μL of a solution of 2 mM pentosidine dissolved in a 100 mM potassium phosphate buffer (pH 8.0) incubated at an arbitrary temperature for 10 minutes were added thereto, and the mixture was stirred and reacted at an arbitrary temperature for 3 minutes. Time-dependent change in A₅₅₅ during the reaction was measured in a spectrophotometer (U-3900, manufactured by Hitachi High-Tech Science Corp.). Time elapsed after the start of measurement—amount of change in A₅₅₅ from 20 seconds to 60 seconds was regarded as an activity value.

The amount of the enzyme that produced 1 μmol of hydrogen peroxide per minute at 37° C. was defined as 1 unit (U). The activity was calculated according to the following expression.

Activity (U/mL)={(ΔAs−ΔA0)×1.6×df}/(39.2×0.5×0.05)

ΔAs: Amount of change in A₅₅₅ per minute of the reaction liquid

ΔA0: Amount of change in A₅₅₅ per minute of the control experiment

1.6: Total volume (mL) of the reaction liquid

df: Dilution coefficient

39.2: Millimolar extinction coefficient (mM⁻¹·cm⁻¹) of a quinonimine dye produced through reaction

0.5: Molar number of a quinonimine dye produced with 1 mol of hydrogen peroxide

0.05: Volume (mL) of the enzyme liquid

The physicochemical properties of penox2 were as follows.

(a) Range of Optimum pH

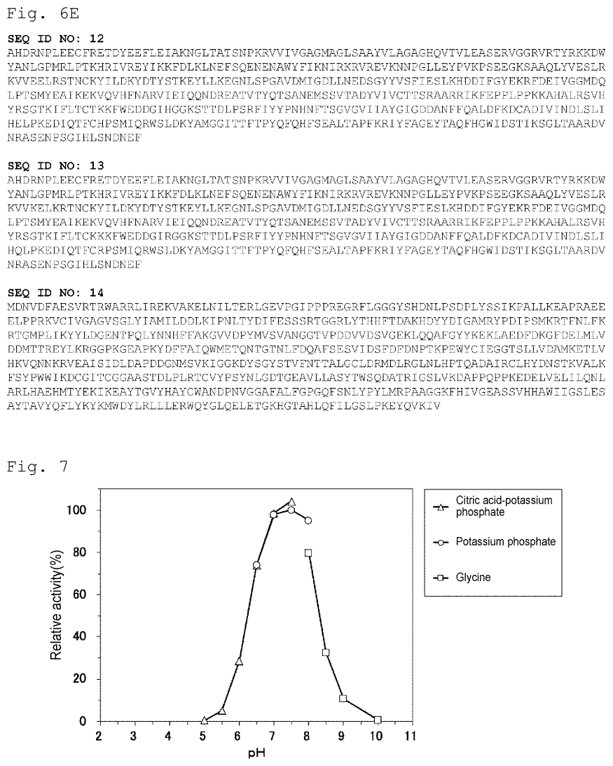

Each buffer was prepared as a 50 mM (final concentration) citric acid-100 mM potassium phosphate buffer (pH 4.0 to 7.5), a 100 mM (final concentration) potassium phosphate buffer (pH 6.5 to 8.0), and a 100 mM (final concentration) glycine buffer (pH 8.0 to 11.0). Enzyme reaction was performed at each pH at a temperature of 37° C. using these buffers. The results are shown in FIG. 7. PenOX2 exhibited the highest activity at pH 7.5. Also, the activity exhibited at pH 6.5 to 8.0 was 70% or more of the activity value around pH 7.5 of the potassium phosphate buffer. It was therefore concluded that the optimum pH of PenOX2 is pH 6.5 to 8.0 and is most preferably pH 7.5.

(b) Range of Optimum Temperature

The activity of PenOX2 was measured at varying temperatures using a 50 mM (final concentration) potassium phosphate buffer (pH 7.5). The results are shown in FIG. 8. The temperature range in which 80% or more activity was exhibited, relative to the activity at a temperature around 50° C. at which the highest activity was exhibited, was from 37° C. to 50° C. It was thus concluded that the range of the optimum temperature of PenOX2 is from 37° C. to 50° C.

(c) Heat Stability

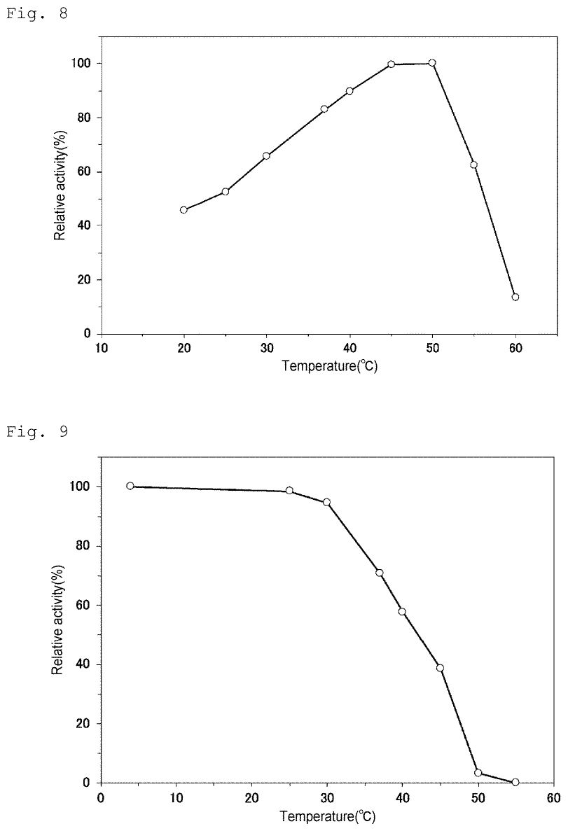

The enzyme liquid treated at each temperature for 10 minutes was evaluated for residual activity by performing the activity measurement described above at a temperature of 37° C. using a 100 mM (final concentration) potassium phosphate buffer (final pH at the time of activity measurement: 7.5). The results about heat stability are as shown in FIG. 9, and PenOX2 was stable up to around 30° C.

(d) Range of Stable pH

Treatment at each pH at 25° C. for 20 hours was performed using a 100 mM citric acid-200 mM potassium phosphate buffer (pH 3.0 to 6.5), a 200 mM potassium phosphate buffer (pH 6.5 to 8.0), or a 200 mM glycine buffer (pH 8.0 to 10.0) as a buffer solution, followed by the measurement of the residual activity of PenOX2. The results are shown in FIG. 10. The pH range in which 90% or more activity was exhibited, relative to the activity of PenOX2 preserved at 4° C., was from pH 4.5 to 7.5, and the pH range in which 60% or more activity was exhibited was from pH 4.0 to 9.0.

(e) Activity Value Against Pentosidine

In the method for measuring activity described above, the activity was measured at 37° C. using a 50 mM (final concentration) potassium phosphate buffer (final pH at the time of activity measurement: 7.5), and an activity value (U/mL) was determined according to the calculation expression described above. The activity value was found to be 7.8 U/mL with specific activity of 29.1 U/mg (Bradford method).

(f) Km Value for Pentosidine

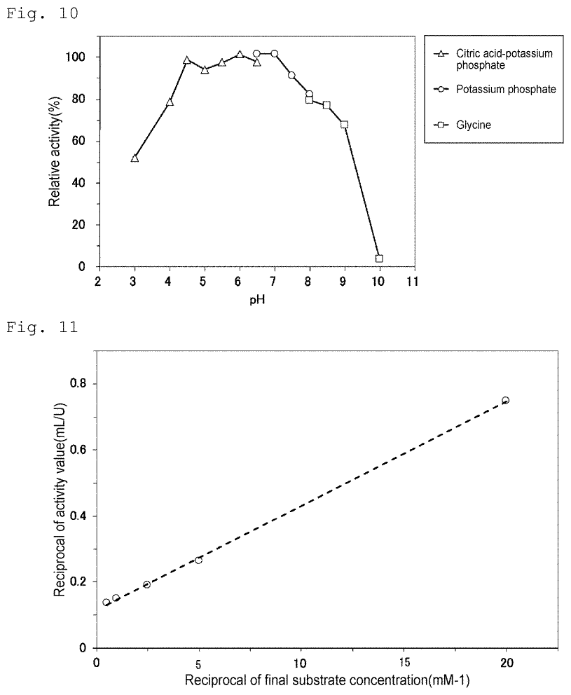

In the method for measuring activity described above, the activity was measured at varying concentrations of the substrate pentosidine at 37° C. using a 50 mM (final concentration) potassium phosphate buffer (pH 7.5), and a Michaelis constant (Km) was determined from a Lineweaver-Burk plot. The results are shown in FIG. 11. The Km value for pentosidine (free form) was found to be 0.070 mM.

(g) Molecular Weight

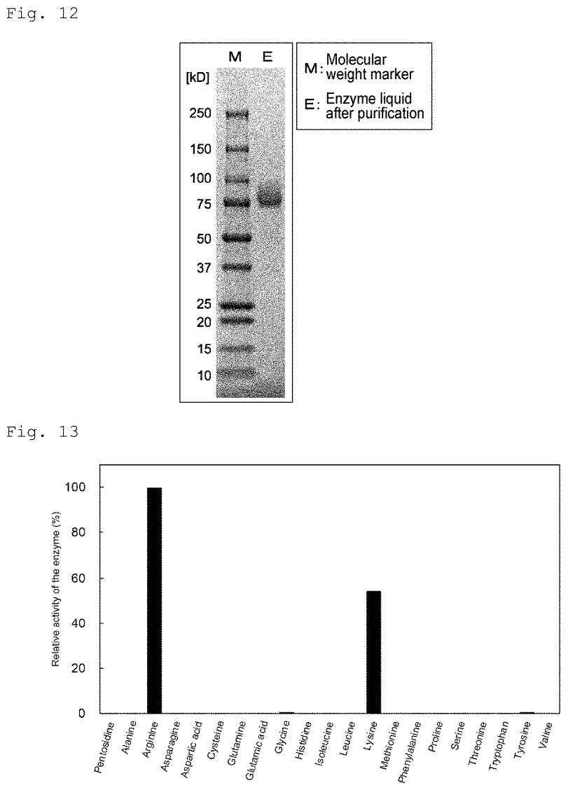

A molecular weight was determined by SDS-PAGE performed according to the method of Laemmli. The electrophoresis gel used was Mini-PROTEAN TGX Stain-Free Precast Gels 4-20% (manufactured by Bio-Rad Laboratories, Inc.), and the molecular weight marker used was Precision Plus Protein All Blue Prestained Protein Standards. The results are shown in FIG. 12. The molecular weight of PenOX2 was approximately 80,000.

### (Example 10) Pentosidine Oxidase Activity Measurement of Enzyme Having Sequence Homology to PenOX1 or PenOX2

As mentioned above, both PenOX1 and PenOX2 had pentosidine oxidase activity. When the percent match between their amino acid sequences was examined using the BLAST program, the amino acid sequence homology therebetween was 38.2%. Subsequently, three enzymes given below were purchased and examined for their pentosidine oxidase activity by the method for measuring activity described above at 37° C. using a 100 mM (final concentration) potassium phosphate buffer (pH 7.5). The amino acid sequence homology to PenOX1 and PenOX2 and pentosidine oxidase activity of each enzyme were as follows.

(a) Crotalus adamanteus-Derived Amino Acid Oxidase Type VI (Manufactured by Merck KGaA) (SEQ ID NO: 12)

### Molecular Weight: 130,000

The amino acid sequence homology of this enzyme to PenOX1 and PenOX2 was 26.8% and 23.5%, respectively. The enzyme was diluted to a concentration of 1 mg/mL (Biuret method) with deionized water and used in activity measurement. The pentosidine oxidase activity of this enzyme was 0.555 (U/mL) with specific activity of 0.555 (U/mg).

(b) Crotalus Atrox-Derived Amino Acid Oxidase Type I (Manufactured by Merck KGaA) (SEQ ID NO: 13)

### Molecular Weight: 59,000 (Calculation Value Based on the Amino Acid Sequence)

The amino acid sequence homology of this enzyme to PenOX1 and PenOX2 was 26.3% and 23.4%, respectively. 1 mg of the enzyme powder was dissolved in 1 mL of deionized water and used in activity measurement. The pentosidine oxidase activity of this enzyme was 0.022 (U/mL) with specific activity of 0.022 (U/mg) as a reference value.

(c) Trichoderma viride-Derived Lysine Oxidase (Manufactured by Merck KGaA) (SEQ ID NO: 14)

### Molecular Weight: 116,000

The amino acid sequence homology of this enzyme to PenOX1 and PenOX2 was 24.0% and 23.3%, respectively. 1 mg of the enzyme powder was dissolved in 1 mL of deionized water and used in activity measurement. The pentosidine oxidase activity of this enzyme was 0.063 (U/mL) with specific activity of 0.063 (U/mg) as a reference value. The sequence homology among the enzymes used in this Example is shown in the following table.

TABLE 2

| | | | (a) | (b) | (c) |
|---|---|---|---|---|---|
| | penox1 | penox2 | Cad_LAAO | Cat_LAAO | Tvi_LysO |
| penox1 | | 38.2% | 26.8% | 26.3% | 24.0% |
| penox2 | | | 23.5% | 23.4% | 23.3% |
| (a) | | | | 98.6% | 25.1% |
| Cad_LAAO | | | | | |
| (b) | | | | | 24.6% |
| Cat_LAAO | | | | | |
| (c) | | | | | |
| Tvi_LysO | | | | | |

### (Example 11) Heterologous Recombinant Expression of Escapin in Aspergillus sojae

The heterologous recombinant expression of a gene encoding mature escapin with a 5′-terminally added gene encoding a signal peptide of the genus Aspergillus was performed with Aspergillus sojae as a host.

Preparation of Expression Vector

The gene sequence of mature escapin used was codon-modified 1,554 base pairs for expression in Aspergillus (SEQ ID NO: 18) based on the amino acid sequence of SEQ ID NO: 15 (nucleotide sequence: SEQ ID NO: 17) derived from Aplysia californica described in the literature (Yang et al., J Exp Biol, 208 (18): 3609-22, 2005).

The gene encoding the signal peptide used was 69-base pair AoCDHss (exon region of a gene encoding the signal peptide of Aspergillus oryzae-derived cellulose dehydrogenase; SEQ ID NO: 16).

In order to integrate the gene encoding mature escapin with the 5′-terminally linked gene encoding a signal peptide of the genus Aspergillus (SEQ ID NO: 16 linked to the 5′ end of SEQ ID NO: 18; hereinafter, referred to as gene sequence A) into a plasmid, gene sequence A with a 5′-terminally added 12-base pair gene sequence (SEQ ID NO: 20) and a 3′-terminally added 12-base pair gene sequence (SEQ ID NO: 21) was obtained (hereinafter, the resulting sequence is referred to as gene sequence A′) by artificial gene synthesis.

For an expression cassette for expressing the gene sequence A, translation elongation factor gene tef1 promoter sequence Ptef (748-bp upstream region of the tef1 gene; SEQ ID NO: 7) was used as a promoter, and alkaline protease gene alp terminator sequence Talp (800-bp downstream region of the alp gene; SEQ ID NO: 8) was used as a terminator.

The selective marker used was transcription marker gene pyrG3 (1,487 bp including a 56-bp upstream region, an 896-bp coding region and a 535-bp downstream region; SEQ ID NO: 9) which complements uracil/uridine auxotrophy and allows multicopy transfer of a gene (see Japanese Patent Laid-Open No. 2018-068292). These Ptef, Talp, and pyrG3 sequences were obtained through PCR reaction with the genomic DNA of Aspergillus sojae NBRC4239 strain as a template.

Next, In-Fusion HD Cloning Kit (manufactured by Clontech Laboratories, Inc.) was used for linking these DNAs. For example, in the case of linking Ptef, the gene sequence A and Talp, DNA fragments were amplified through PCR reaction using the reverse primer of SEQ ID NO: 22 for Ptef and the forward primer of SEQ ID NO: 23 for Talp. In this respect, the 5′ end of the gene sequence A′ had a 15-bp sequence (CAT sequence complementary to the start codon ATG of AoCDHss, and SEQ ID NO: 20) complementary to the 5′ end of the reverse primer for Ptef amplification (SEQ ID NO: 22). The 3′ end of the gene sequence A′ had a 15-bp sequence (the stop codon TGA sequence of escapin and SEQ ID NO: 21) complementary to the 5′ end of the forward primer for Talp amplification (SEQ ID NO: 23). Therefore, Ptef, the gene sequence A and Talp can be linked through in-fusion reaction. In this way, expression vector p19-pG3-AoCDHss-Escapin was prepared in which Ptef-AoCDHss-Escapin-Talp-pyrG3 consisting of Ptef, AoCDHss, mature escapin, Talp and pyrG3 linked in this order was inserted in the multicloning site of a pUC19 plasmid.

Preparation and Culture of Expressing Aspergillus Strain

A pyrG gene disruptant (strain deficient in a 48-bp upstream region, an 896-bp coding region and a 240-bp downstream region of the pyrG gene) of Aspergillus sojae was transformed by the protoplast PEG method using the plasmid p19-pG3-AoCDHss-Escapin for transformation obtained as described above to obtain two AoCDHss-Escapin strain as Aspergillus sojae transformants having multicopy inserts of the expression cassette of AoCDHss-Escapin.

Each of the obtained Aspergillus sojae transformants (AoCDHss-Escapin strain) was inoculated to 15 mL of PPY liquid medium (2% (w/v) Pinedex, 1% (w/v) Polypeptone, 0.5% (w/v) yeast extracts, 0.5% (w/v) monopotassium dihydrogen phosphate, and 0.05% (w/v) magnesium sulfate heptahydrate) contained in a 50 mL Erlenmeyer flask, and shake-cultured at 30° C. for 4 to 5 days.

### (Example 12) Measurement of Pentosidine in Combination with Amino Acid Degrading Enzyme

Preparation of Solution Containing Amino Acid Degrading Enzyme 1 Liquid (Escapin; SEQ ID NO: 15)

The culture supernatant of each transformant obtained in Example 11 was concentrated using an ultrafiltration membrane (Amicon Ultra 15-30k, manufactured by Merck KGaA).

A saturated aqueous solution of ammonium sulfate cooled in ice was added to the concentrate cooled in ice so as to attain 70% saturation of ammonium sulfate. The mixture was left at 4° C. for 2 hours and then centrifuged (15,000 rpm, 4° C., 15 min) to recover a supernatant, which was then redissolved in a 0.1 M potassium phosphate buffer (pH 6.8).

Preparation of Amino Acid Degrading Enzyme 2 Liquid (Solution Containing Crotalus adamanteus-Derived L-Amino Acid Oxidase (SEQ ID NO: 19))

Crotalus adamanteus-derived L-amino acid oxidase Type I (L-Amino Acid Oxidase from Crotalus adamanteus, Type I, manufactured by Merck KGaA) was dissolved at 1 mg/ml in a 0.1 M potassium phosphate buffer (pH 6.8). This solution was concentrated using an ultrafiltration membrane (Amicon Ultra 15-30 k, manufactured by Merck KGaA).

Preparation of Enzyme Liquid for Pentosidine Measurement

The A. sojae recombinant strain As-penox2 obtained in the sections “Preparation of expression vector” and “Preparation and culture of expressing Aspergillus strain” in Example 5 was shake-cultured at 180 rpm at 30° C. for 5 days in sterilized PPY medium. The obtained culture supernatant was concentrated using an ultrafiltration membrane (Amicon Ultra 15-30k, manufactured by Merck KGaA). Ammonium sulfate was gradually added to the concentrate so as to attain 70% saturation. The mixture was left at 4° C. for 2 hours and then centrifuged (15,000 rpm, 4° C., 15 min) to recover a supernatant. The recovered supernatant was concentrated using an ultrafiltration membrane (Amicon Ultra 0.5-30k, manufactured by Merck KGaA) and buffer-replaced with a 0.1 M potassium phosphate buffer (pH 6.8).

Foreign Substance Elimination Test in Pentosidine Measurement

A model system involving various amino acids artificially added as foreign substances to a solution to be measured was used as a pentosidine measurement system using pentosidine oxidase to verify the effect of the measurement method of the present invention on pentosidine measurement.

(1) Preparation of Pentosidine Solution

Pentosidine (in terms of a free form) (manufactured by Peptide Institute, Inc.; 3TFA salt was used) was dissolved at 0.1 mM, 0.2 mM, 0.3 mM, 0.4 mM or 0.5 mM in deionized water.

(2) Preparation of Foreign Substance Solution

L-Alanine (290 μM), L-cysteine (48 μM), L-aspartic acid (4 μM), L-glutamic acid (57 μM), L-phenylalanine (40 μM), glycine (245 μM), L-isoleucine (54 μM), L-lysine (128 μM), L-leucine (92 μM), L-methionine (22 μM), L-proline (184 μM), L-arginine (60 μM), L-serine (94 μM), L-threonine (116 μM), L-valine (155 μM), L-tryptophan (39 μM) and L-tyrosine (45 μM) were each dissolved in a 0.1 M (final concentration) potassium phosphate buffer (pH 6.8). The amino acid concentrations were established with reference to each data (values of half the upper limit values) on the amounts of the amino acids in the blood of 18-old-year or older people in the values of the literature (Mayo Clinic Laboratories Neurology Catalog, “Plasma Amino Acid Reference Values” (https://neurology.testcatalog.org/show/AAQP, date of access: Oct. 10, 2018; https://www.mayomedicallaboratories.com/test-catalog/Clinical+and+Interpretive/9265, date of access: Jun. 24, 2020).

(3) Preparation of Reagent

Reagents for use in measurement were prepared as follows.

3A. Coloring Reagent

Peroxidase (manufactured by Toyobo Co., Ltd.) (3.99 U/ml), 4-aminoantipyrine (manufactured by Fluka/Honeywell International Inc.) (1.8 mM), and TOOS (manufactured by Dojindo Laboratories Co., Ltd.) (2 mM) were dissolved in deionized water.

3B. Foreign Substance Elimination Reagent

The amino acid degrading enzyme 1 liquid (1.63 U/ml) and the amino acid degrading enzyme 2 liquid (2.40 U/ml) were mixed at a ratio of 5:3 in terms of the amounts of the liquids.

3C. Reagent for Pentosidine Measurement

The enzyme liquid for pentosidine measurement (3.31 U/ml) was used.

(4) Measurement

Every measurement was performed at room temperature unless otherwise specified. A 96-well microwell plate (manufactured by Nunc/Thermo Fisher Scientific, Inc.) was used in reaction. 20 μl of the foreign substance solution of (2), 25 μl of the coloring reagent of (3A), and 50 μl of the foreign substance elimination reagent of (3B) were added in order to 5 μl of the pentosidine solution of (1). Ten minutes later, absorbance at 555 nm was measured and designated as data 1.

Subsequently, 25 μl of the reagent for pentosidine measurement of (3C) was added thereto. Ten minutes later, absorbance at 555 nm was measured and designated as data 2. A value (ΔOD) obtained by subtracting the data 1 from the data 2 was used as a measurement value. In Comparative Example 1, measurement was also performed using a solution free from foreign substances, i.e., a solution containing a 0.1 M potassium phosphate buffer (pH 6.8) replaced for the foreign substance solution of (2).

In Comparative Example 2, measurement was also performed using a system involving foreign substances without foreign substance elimination, i.e., a solution containing a 0.1 M potassium phosphate buffer (pH 6.8) replaced for the foreign substance elimination reagent of (3B).

Table 3 given below shows an average value from measurement performed three times. Table 4 shows relative values of Comparative Example 2 and Example wherein the measurement value of Comparative Example 1 was defined as 100%.

TABLE 3

| | | Comparative | Comparative | Example |
|---|---|---|---|---|
| | | Example 1 | Example 2 | 12 |
| Presence or absence of foreign | | Absent | Present | Present |
| substance addition | | | | |
| Presence or absence of foreign | | Present | Absent | Present |
| substance elimination reagent | | | | |
| addition | | | | |
| Pentosidine | 5 nM | 0.067 | 0.203 | 0.089 |
| concentration | 10 nM | 0.120 | 0.239 | 0.128 |
| | 15 nM | 0.115 | 0.280 | 0.138 |
| | 20 nM | 0.160 | 0.304 | 0.175 |
| | 25 nM | 0.191 | 0.329 | 0.186 |

TABLE 4

| | | Comparative | Comparative | Example |
|---|---|---|---|---|
| | | Example 1 | Example 2 | 12 |
| Presence or absence of foreign | | Absent | Present | Present |
| substance addition | | | | |
| Presence or absence of foreign | | Present | Absent | Present |
| substance elimination reagent | | | | |
| addition | | | | |
| Pentosidine | 5 nM | 100% | 303% | 133% |
| concentration | 10 nM | 100% | 199% | 107% |
| | 15 nM | 100% | 243% | 120% |
| | 20 nM | 100% | 190% | 109% |
| | 25 nM | 100% | 172% | 103% |

As shown in the tables, in pentosidine measurement without foreign substance elimination (Comparative Example 2), much higher pentosidine concentrations which reflected the addition of foreign substances were measured than those in measurement without foreign substance addition (Comparative Example 1), and accurate values were not obtained.

By contrast, in measurement comprising the elimination step using the foreign substance elimination reagent, measurement values ascribable to foreign substances were reduced, and values close to the pentosidine concentrations obtained in the foreign substance-free system of Comparative Example 1 were obtained. Thus, accurate measurement was achieved by reducing the influence of foreign substances leading to measurement errors.

### (Example 13) Substrate Specificity Analysis of Amino Acid Degrading Enzyme 1

The amino acid eliminating enzyme 1 was analyzed for its substrate specificity for various amino acids and pentosidine.

(1) Preparation of Enzyme Liquid

The amino acid eliminating enzyme 1 liquid obtained in Example 12 was diluted to 0.134 U/ml with a 0.1 M potassium phosphate buffer (pH 6.8).

(2) Preparation of Various Amino Acid and Pentosidine Solutions

Pentosidine (which was the same as in Example 12) was dissolved at 2 mM in deionized water. Various amino acids were each dissolved at 4 mM in deionized water.

(3) Preparation of Coloring Reagent

Peroxidase (manufactured by Toyobo Co., Ltd.) (3.99 U/ml), 4-aminoantipyrine (manufactured by Fluka/Honeywell International Inc.) (1.8 mM), and TOOS (manufactured by Dojindo Laboratories Co., Ltd.) (2 mM) were dissolved in deionized water.

(4) Measurement

Every measurement was performed at room temperature unless otherwise specified. A 96-well microwell plate (manufactured by Nunc/Thermo Fisher Scientific, Inc.) was used in reaction.

25 μl of each amino acid solution or the pentosidine solution of (2) and 25 μl of the coloring reagent of (3) were added in order to 50 μl of the enzyme liquid of (1). At the start and ten minutes later, absorbance at 555 nm was measured. A slope of elevation in absorbance was regarded as a reaction rate for the target substrate.

FIG. 13 shows reaction rates for various amino acids and pentosidine wherein the reaction rate for arginine which was the substrate having the highest reaction rate was defined as 100, i.e., substrate specificity.

### (Example 14) Substrate Specificity Analysis of Amino Acid Eliminating Enzyme 2

The amino acid eliminating enzyme 2 was analyzed for its substrate specificity for various amino acids and pentosidine.

(1) Preparation of enzyme liquid

The amino acid eliminating enzyme 2 liquid obtained in Example 12 was diluted to 0.12 U/ml with a 0.1 M potassium phosphate buffer (pH 6.8).

Various amino acid and pentosidine solutions (2) and a coloring reagent (3) were prepared in the same way as in Example 13.

(4) Measurement

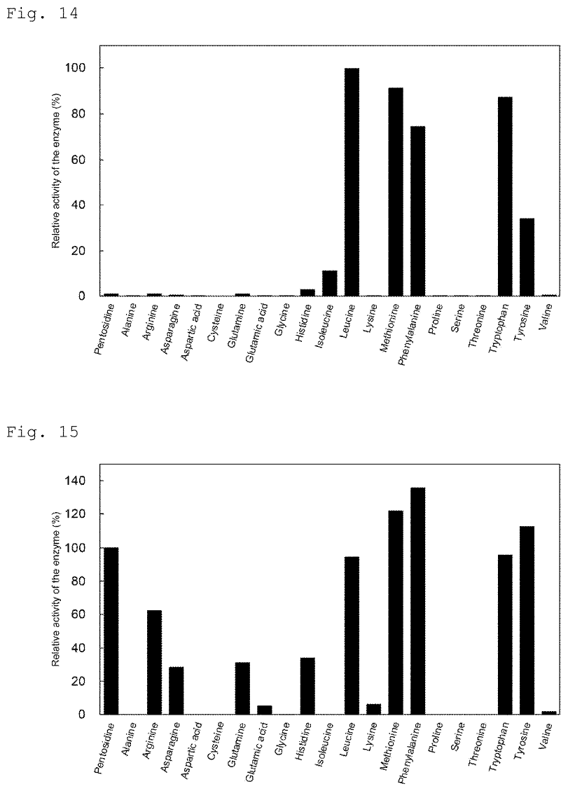

Every measurement was performed at room temperature unless otherwise specified. A 96-well microwell plate (manufactured by Nunc/Thermo Fisher Scientific, Inc.) was used in reaction. 25 μl of each amino acid solution or the pentosidine solution of (2) and 25 μl of the coloring reagent of (3) were added in order to 50 μl of the enzyme liquid of (1). At the start and ten minutes later, absorbance at 555 nm was measured. A slope of elevation in absorbance was regarded as a reaction rate for the target substrate. FIG. 14 shows reaction rates for various amino acids and pentosidine wherein the reaction rate for leucine which was the substrate having the highest reaction rate was defined as 100, i.e., substrate specificity.

### (Example 15) Substrate Specificity Analysis of Enzyme for Pentosidine Measurement

The enzyme for pentosidine measurement was analyzed for its substrate specificity for various amino acids and pentosidine.

(1) Preparation of Enzyme Liquid

The enzyme liquid for pentosidine measurement obtained in Example 12 was diluted to 0.083 U/ml with a 0.1 M potassium phosphate buffer (pH 6.8).

Various amino acid and pentosidine solutions (2) and a coloring reagent (3) were prepared in the same way as in Example 13.

(4) Measurement

Every measurement was performed at room temperature unless otherwise specified. A 96-well microwell plate (manufactured by Nunc/Thermo Fisher Scientific, Inc.) was used in reaction. 25 μl of each amino acid solution or the pentosidine solution of (2) and 25 μl of the coloring reagent of (3) were added in order to 50 μl of the enzyme liquid of (1). At the start and ten minutes later, absorbance at 555 nm was measured. A slope of elevation in absorbance was regarded as a reaction rate for the target substrate.

FIG. 15 shows reaction rates, for various amino acids wherein the reaction rate for pentosidine was defined as 100 i.e., substrate specificity.

The results of Examples 13 to 15 suggested that in Example 12, amino acids highly reactive with the enzyme for pentosidine measurement were eliminated by the amino acid degrading enzyme 1 liquid and the amino acid degrading enzyme 2 liquid, and the accurate measurement of pentosidine was achieved by reducing the influence of foreign substances leading to measurement errors.

1-12. (canceled)

13. A method for measuring pentosidine in a specimen, the measurement method comprising the steps of:
degrading the specimen with an amino acid degrading enzyme;
contacting the specimen after the degradation step with a protein having activity that oxidatively degrades pentosidine; and
detecting change resulting from the contact, wherein
the amino acid degrading enzyme and the protein having activity that oxidatively degrades pentosidine are different from each other.

14. The measurement method according to claim 13, wherein in the detection step, change in an amount of oxygen, hydrogen peroxide or ammonia is detected.

15. The measurement method according to claim 13, wherein the protein having activity that oxidatively degrades pentosidine has the following physicochemical properties:
(1) action: activity that oxidatively degrades pentosidine; and
(2) molecular weight based on SDS-PAGE: 75,000 to 85,000.

16. The measurement method according to claim 14, wherein the protein having activity that oxidatively degrades pentosidine has the following physicochemical properties:
(1) action: activity that oxidatively degrades pentosidine; and
(2) molecular weight based on SDS-PAGE: 75,000 to 85,000.

17. The measurement method according to claim 13, wherein the protein having activity that oxidatively degrades pentosidine is any protein selected from the group consisting of the following (a) to (f):
(a) a protein consisting of the amino acid sequence as set forth in SEQ ID NO: 2 or SEQ ID NO: 4;
(b) a protein encoded by a gene consisting of the nucleotide sequence as set forth in SEQ ID NO: 1, SEQ ID NO: 3, SEQ ID NO: 5 or SEQ ID NO: 6;
(c) a protein consisting of an amino acid sequence having 75% or higher identity to the amino acid sequence as set forth in SEQ ID NO: 2 or SEQ ID NO: 4;
(d) a protein encoded by a gene consisting of a nucleotide sequence having 75% or higher identity to the nucleotide sequence as set forth in SEQ ID NO: 1, SEQ ID NO: 3, SEQ ID NO: 5 or SEQ ID NO: 6;
(e) a protein consisting of an amino acid sequence derived from the amino acid sequence as set forth in SEQ ID NO: 2 or SEQ ID NO: 4 by the deletion, substitution and/or addition of one or more amino acids; and
(f) a protein encoded by a nucleotide sequence that hybridizes under stringent conditions to the nucleotide sequence as set forth in SEQ ID NO: 1, SEQ ID NO: 3, SEQ ID NO: 5 or SEQ ID NO: 6.

18. The measurement method according to claim 13, wherein the protein having activity that oxidatively degrades pentosidine is derived from a filamentous fungus.

19. The measurement method according to claim 14, wherein the protein having activity that oxidatively degrades pentosidine is derived from a filamentous fungus.

20. The measurement method according to claim 15, wherein the protein having activity that oxidatively degrades pentosidine is derived from a filamentous fungus.

21. The measurement method according to claim 16, wherein the protein having activity that oxidatively degrades pentosidine is derived from a filamentous fungus.

22. The measurement method according to claim 17, wherein the protein having activity that oxidatively degrades pentosidine is derived from a filamentous fungus.

23. The measurement method according to claim 13, wherein
the protein having activity that oxidatively degrades pentosidine is pentosidine oxidase, and
the amino acid degrading enzyme degrades an amino acid contained in the specimen, wherein the amino acid is selected from arginine, leucine, methionine, phenylalanine, tryptophan and tyrosine.

24. The measurement method according to claim 13, wherein the amino acid to be degraded by the amino acid degrading enzyme is an amino acid against which the protein having activity that oxidatively degrades pentosidine has 40% or higher relative activity when the activity of the protein having activity that oxidatively degrades pentosidine against pentosidine is defined as 100%.

25. The measurement method according to claim 13, wherein the amino acid degrading enzyme is selected from the group consisting of amino acid oxidase, amino acid dehydrogenase, amino acid aminotransferase, amino acid decarboxylase, amino acid ammonia lyase, amino acid oxygenase and amino acid hydrolase.

26. A kit for measuring pentosidine in a specimen, comprising:
(i) an amino acid degrading enzyme; and
(ii) a protein having activity that oxidatively degrades pentosidine.

27. The kit according to claim 26, wherein the protein having activity that oxidatively degrades pentosidine is any protein selected from the group consisting of the following (a) to (f):
(a) a protein consisting of the amino acid sequence as set forth in SEQ ID NO: 2 or SEQ ID NO: 4;
(b) a protein encoded by a gene consisting of the nucleotide sequence as set forth in SEQ ID NO: 1, SEQ ID NO: 3, SEQ ID NO: 5 or SEQ ID NO: 6;
(c) a protein consisting of an amino acid sequence having 75% or higher identity to the amino acid sequence as set forth in SEQ ID NO: 2 or SEQ ID NO: 4;
(d) a protein encoded by a gene consisting of a nucleotide sequence having 75% or higher identity to the nucleotide sequence as set forth in SEQ ID NO: 1, SEQ ID NO: 3, SEQ ID NO: 5 or SEQ ID NO: 6;
(e) a protein consisting of an amino acid sequence derived from the amino acid sequence as set forth in SEQ ID NO: 2 or SEQ ID NO: 4 by the deletion, substitution and/or addition of one or more amino acids; and
(f) a protein encoded by a nucleotide sequence that hybridizes under stringent conditions to the nucleotide sequence as set forth in SEQ ID NO: 1, SEQ ID NO: 3, SEQ ID NO: 5 or SEQ ID NO: 6.

28. The kit according to claim 26, wherein the amino acid degrading enzyme is an enzyme that degrades an amino acid selected from arginine, leucine, methionine, phenylalanine, tryptophan and tyrosine.

29. The kit according to claim 27, wherein the amino acid degrading enzyme is an enzyme that degrades an amino acid selected from arginine, leucine, methionine, phenylalanine, tryptophan and tyrosine.

30. A method for producing a reaction product of pentosidine derived from a specimen, the method comprising the steps of:
degrading the specimen with an amino acid degrading enzyme; and
contacting the specimen after the degradation step with a protein having activity that oxidatively degrades pentosidine, wherein
the amino acid degrading enzyme and the protein having activity that oxidatively degrades pentosidine are different from each other.
